(19) United States
(12) Patent Application Publication
(10) Pub. No.: US 2022/0195474 A1
(43) Pub. Date: Jun. 23, 2022

(54) METABOLIC ENGINEERING OF NON-PATHOGENIC ESCHERICHIA COLI STRAINS FOR THE CONTROLLED PRODUCTION OF LOW MOLECULAR WEIGHT HEPAROSAN AND SIZE-SPECIFIC HEPAROSAN OLIGOSACCHARIDES

(71) Applicant: University of Utah Research Foundation, Salt Lake City, UT (US)

(72) Inventors: Anindita ROY, Salt lake City, UT (US); Yuma MIYAI, Richmond, VA (US); Allesandro ROSSI, Salt Lake City, UT (US); Ulmesh DESAI, Glen Allen, VA (US); Yukio SAIJOH, Salt Lake City, UT (US); Kuberan BALAGURUNATHAN, Salt Lake City, UT (US)

(21) Appl. No.: 17/594,643

(22) PCT Filed: Apr. 24, 2020
(86) PCT No.: PCT/US2020/029902

§ 371 Date: Oct. 25, 2021

Related U.S. Application Data
(60) Provisional application No. 62/838,432, filed on Apr. 25, 2019.
(60) Provisional application No. 62/914,112, filed on Oct. 11, 2019.

(51) Int. Cl. C12P 19/04; C12N 15/52; C12P 19/18; C12N 1/20

(52) U.S. Cl. CPC C12P 19/04; C12N 15/52; C12R 2001/19; C12N 1/205; C12P 19/18

(57) ABSTRACT

Methods for producing heparosan oligosaccharides and polysaccharides include culturing a recombinant host cell of a non-pathogenic E. coli strain, the host cell having been engineered to comprise the biosynthetic gene cluster kfiA, KfiB, kfiC, kfiD and, optionally, the gene elmA in culture conditions enabling direct expression heparosan oligosaccharides of specific sizes and/or of heparosan low molecular weight precursors by the host cell. The methods further includes obtaining such expression products during the culturing. A majority of the heparosan oligosaccharides range in size from approximately tetrasaccharide to approximately dodecasaccharide and a majority of the heparosan polysaccharides range in mass from approximately 5 KDa to approximately 30 KDa.

### CROSS-REFERENCE TO RELATED APPLICATIONS

This application is a National Phase of PCT Application No. PCT/US2020/029902, filed on Apr. 24, 2020, and entitled METABOLIC ENGINEERING OF NON-PATHOGENIC ESCHERICHIA COLI STRAINS FOR THE CONTROLLED PRODUCTION OF LOW MOLECULAR WEIGHT HEPAROSAN AND SIZE-SPECIFIC HEPAROSAN OLIGOSACCHARIDES, the entirety of which is hereby incorporated by reference, which claims the benefit of U.S. Provisional Application No. 62/914,112, filed Oct. 11, 2019 and entitled METABOLIC ENGINEERING OF NON-PATHOGENIC ESCHERICHIA COLI STRAINS FOR THE CONTROLLED PRODUCTION OF LOW MOLECULAR WEIGHT HEPAROSAN AND SIZE-SPECIFIC HEPAROSAN OLIGOSACCHARIDES, the entirety of which is hereby incorporated by reference, and U.S. Provisional Application No. 62/838,432, filed Apr. 25, 2019 and entitled RECOMBINANT LOW MOLECULAR WEIGHT HEPAROSAN AND HEPAROSAN OLIGOSACCHARIDES FROM NON-PATHOGENIC BACTERIA, the entirety of which is hereby incorporated by reference.

### STATEMENT REGARDING FEDERALLY SPONSORED RESEARCH OR DEVELOPMENT

This invention was made with government support under Grant No. HL107512, awarded by the National Institutes of Health. The government has certain rights in the invention.

### BACKGROUND OF THE INVENTION

### Field of the Invention

This invention relates to the production of heparosan generally and more specifically to methods and systems for the controlled production of low molecular weight heparosan and size-specific heparosan oligosaccharides.

### Description of the Related Art

Heparin, a lifesaving blood thinner used in over 100 million surgical procedures worldwide annually, is currently isolated from over 700 million pigs and ˜200 million cattle in slaughterhouses worldwide. Though animal-derived heparin has been in use over eight decades, it is a coraplex mixture that poses a risk for chemical adulteration, and its availability is highly vulnerable. Therefore, there is an urgent need in devising bioengineering approaches for the production of heparin polymers, especially low molecular weight heparin (LMWH), and thus, relying less on animal sources. One of the main challenges, however, is the rapid, cost effective production of low molecular weight heparosan, a precursor of LMWH, and size-defined heparosan oligosaccharides.

Glycosaminoglycans (GAGs) are structurally diverse, highly anionic linear polysaccharides. They play critical roles in various developmental, physiological, and pathological processes. GAGs are comprised of repeating disaccharide units that differ in terms of sugar building blocks, anomeric linkages, and modifications. Heparan sulfate (HS) and heparin are two members of the GAG family that regulate numerous biological processes such as cell proliferation and differentiation, cell-cell interactions and adhesion, receptor-mediated interaction with proteins and pathogens, angiogenesis, blood coagulation, neuronal development, and cancer metastasis among others. Though heparin is predicted to confer benefits in treating many human diseases, it has been primarily exploited therapeutically for its anticoagulant activity in various medical procedures since the 1940s. Heparins, including unfractionated heparin (UFH) and LMWHs, are the most commonly used clinical anticoagulants with an estimated annual market cap of around 2 to 5 billion dollars. Full-length heparin and HS, LMWHs, and oligosaccharides derived from them are highly researched agents with potential use in several diseases.

HS is found extensively in the extracellular matrix both by itself or linked to a serine residues of certain proteins called heparan sulfate proteoglycans (HSPGs). HSPGs are membrane-anchored, cell-surface molecules that can function as cell receptors or cell identifiers. Both HS and heparin have similar disaccharide repeating units of a D-glucosamine (GlcN) α-(1→4) linked to a hexuronic acid, D-glucuronic acid (GlcA) or L-iduronic Acid (IdoA). The GlcN residues can carry modifications such as N-acetyl (GlcNAc) or N-sulfate (GlcNS), 3-O- and 6-O-sulfate groups whereas the GlcA/IdoA residues may carry a 2-O-sulfate group. These modifications are orchestrated in the Golgi apparatus by a large number of biosynthetic enzymes and their isoforms: N-Deacetylase/N-sulfotransferase (NDST), C5 epimerase, 2-O-sulfotransferase (2OST), 3-O-sulfotransferase (3OST), and 6-O-sulfotransferase (6OST). These modifications contribute to high polydispersity and dictate heparin-protein interactions, which in turn fine tune diverse biological functions. However, this enormous structural diversity also creates significant hurdles to the study of the structure-function relationships of heparin-protein interactions.

At present, clinically used low molecular weight heparin anticoagulants are obtained from UHF polymers extracted from animal sources, mostly from bovine lungs and porcine intestines. Despite extensive purification and characterization of heparin extracts, animal sources still pose several risks, including heparin adulteration, as happened during the 2008 heparin crisis. Also, risks such as deadly African Swine Fever may cause reduction of pig population worldwide leading to a potentially severe shortage of heparin anticoagulants. Though chemical synthesis of anticoagulantly-active heparin polymer is challenging, this approach was implemented in the large scale production of heparin pentasaccharide that is currently available as a commercial drug. Further, animal-derived UHF carries the possibility of tissue-specific and gene-pool specific variability in the composition of raw material used to prepare UHF. Moreover, it is nearly impossible to isolate a heparin chain of a specific length directly from an animal source. Heparin sourced from animals is a highly complex mixture of sulfated polysaccharides of varying lengths and sulfation patterns and only a third of the mixture is pharmacologically active.

To counter these problems, chemical synthesis has been pursued. However, this is time consuming, complicated, and extremely expensive. As of now, only short oligosaccharides, especially di- and tetra-saccharides, have been synthesized, which typically are unable to function as well as UFH. Longer sequences, e.g., hexa- and octa-saccharides, have been synthesized, though their synthesis is case-specific and not amenable for application to a wide range of sequences.

Chemoenzymatic synthesis, on the other hand, involves the use of various biologically-sourced biosynthetic enzymes to rapidly assemble specific heparin structures. Although the chemoenzymatic approach offers great promise in the production of larger heparin structures, there are several challenges involved, including the cost-effective production of heparosan, the backbone precursor structure of heparin. Heparosan polysaccharide consists of repeating disaccharide units of [-GlcA-β-(1→4)-GlcNAc-α-(1→4)-], isolated from the bacterium E. coli K5. This bacterium has a K5 biosynthetic gene cluster comprising a heparosan biosynthetic operon. However, the K5 E. coli strain is a human pathogen that can cause severe urinary tract infections, which renders it undesirable for use in heparosan production.

Cloning the second region of the K5 biosynthetic gene cluster, specifically the region KfiABCD, into non-pathogenic bacterial strains has been shown to produce recombinant heparosan polymers. Yet, the relevance of different plasmid constructs and/or bacterial strains on the heparosan formed in the culture may be better defined, and previous reports have suggested only the production of very high molecular weight heparosan, ranging from ˜25,000 Da to ˜150,000 Da in this manner, which may not be suitable as a substrate in the production of heparin structures for clinical usage, as UHF has lower predictable pharmacokinetic properties compared to LMWH. Conventional attempts at preparing size-specific oligosaccharides from polysaccharides, through partial digestion using recombinant heparin lyases, are inefficient as heparin lyases cleave the polymer through processive exolytic mechanism resulting predominantly in the generation of disaccharides. However, the minimum size of heparin, which is required for binding to antithrombin III and to function as an anticoagulant, is a pentasaccharide. Additionally, for the study of the structure-function relationship of heparin-like structures with various proteins, production of size- and sulfation-specific oligosaccharides are required.

Thus, there is an urgent need for the production of LMWH (˜3 to ˜5 KDa) that will overcome at least one major hurdle in the quest for production of biotechnological LMWHs for clinical usage.

Various embodiments of the several inventions disclosed herein address these, inter alia, issues.

### SUMMARY

It would be desirable to devise a strategy to produce LMWH (˜3 to 5 KDa) and heparosan oligosaccharides in non-pathogenic bacterial strains (e.g., non-pathogenic E. coli strains). As discussed herein, it has been determined that K5 eliminase, an enzyme encoded by the eliminase (elmA) gene in E. coli K5, specifically generates heparosan oligosaccharides of a desirably larger size than the disaccharides achieved by partial digestion with recombinant heparin lyases.

To this end, methods and systems for engineering non-pathogenic E. coli strains by transformation with the essential heparosan biosynthetic genes from pathogenic E. coli K5, with or without elmA, and producing LMWH and a range of size-specific heparosan oligosaccharides with such transformed strains in a controlled manner through modulating culture conditions, are described herein. The methods and systems described herein are promising and will pave the way for large scale production of LMANH anticoagulants in the future.

An embodiment is a method for producing heparosan oligosaccharides, the method comprising: culturing a recombinant host cell of a non-pathogenic E. coli strain, the recombinant host cell having been engineered to comprise the biosynthetic gene cluster KfiA, KfiC, KfiD and the gene elmA, in culture conditions enabling direct expression of heparosan oligosaccharides by the recombinant host cell; and obtaining a plurality of heparosan oligosaccharides expressed by the recombinant host cell during the culturing, wherein a majority of the plurality of heparosan oligosaccharides range in size from approximately tetrasaccharide to approximately dodecasaccharide.

A further embodiment is a recombinant host cell of a non-pathogenic E. coli strain, the recombinant host cell having been engineered to comprise the biosynthetic gene cluster KfiA, KfiB, KfiC, KfiD and the gene elmA, wherein the recombinant host cell is engineered to directly express a plurality of heparosan oligosaccharides when cultured under suitable conditions, and wherein a majority of the plurality of heparosan oligosaccharides range in size from approximately tetrasaccharide to approximately dodecasaccharide.

A further embodiment is a method for producing heparosan polysaccharides, the method comprising: culturing a recombinant host cell of a non-pathogenic E. coli strain, the recombinant host cell having been engineered to comprise the biosynthetic gene cluster KfiA, KfiB, KfiC, in culture conditions enabling direct expression of heparosan polysaccharides by the recombinant host cell; and obtaining a plurality of heparosan polysaccharides expressed by the recombinant host cell during the culturing, wherein a majority of the plurality of heparosan polysaccharides range in mass from approximately 5 KDa to approximately 30 KDa.

A further embodiment is a recombinant host cell of a non-pathogenic E. coli strain, the recombinant host cell having been engineered to comprise the biosynthetic gene cluster KfiA, KfiB, KfiC, KfiD, wherein the recombinant host cell is engineered to directly express a plurality of heparosan polysaccharides when cultured under suitable conditions, and wherein a majority of the plurality of heparosan polysaccharides range in size from approximately 5 KDa to approximately 30 KDa.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

### DETAILED DESCRIPTION OF THE DRAWINGS

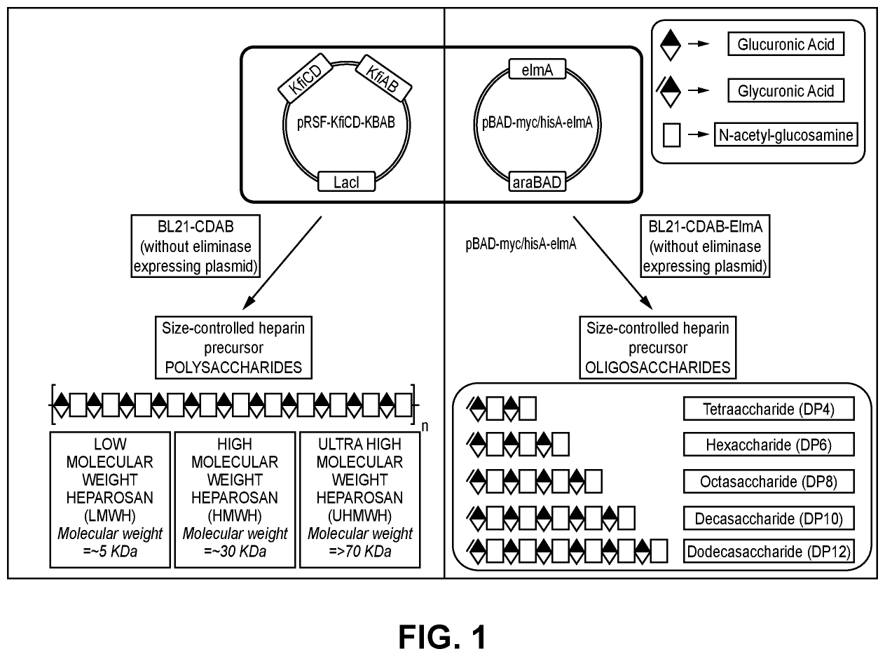

FIG. 1 is a graphical representation of an overview of the production of heparosan LMWH precursors and heparosan oligosaccharides of specific sizes.

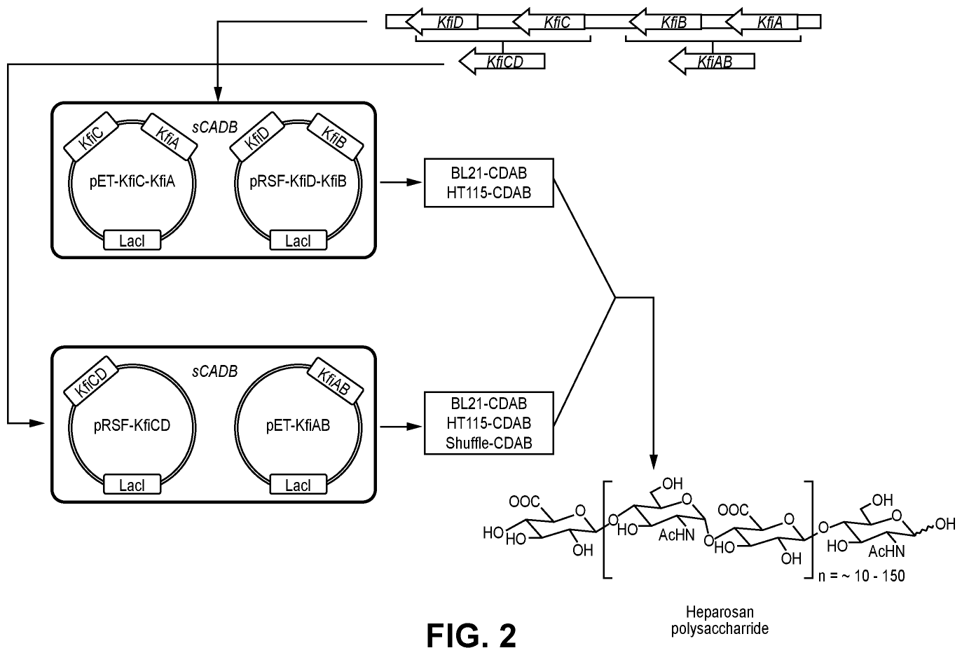

FIG. 2 is a graphical representation of the cloning strategy of the different combinations of KfiABCD gene constructs and bacterial strains for heparosan production.

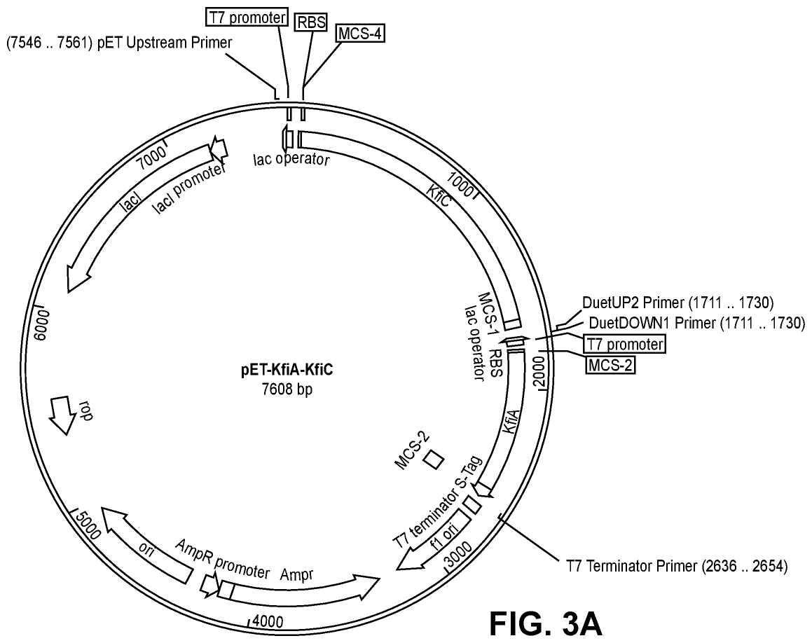

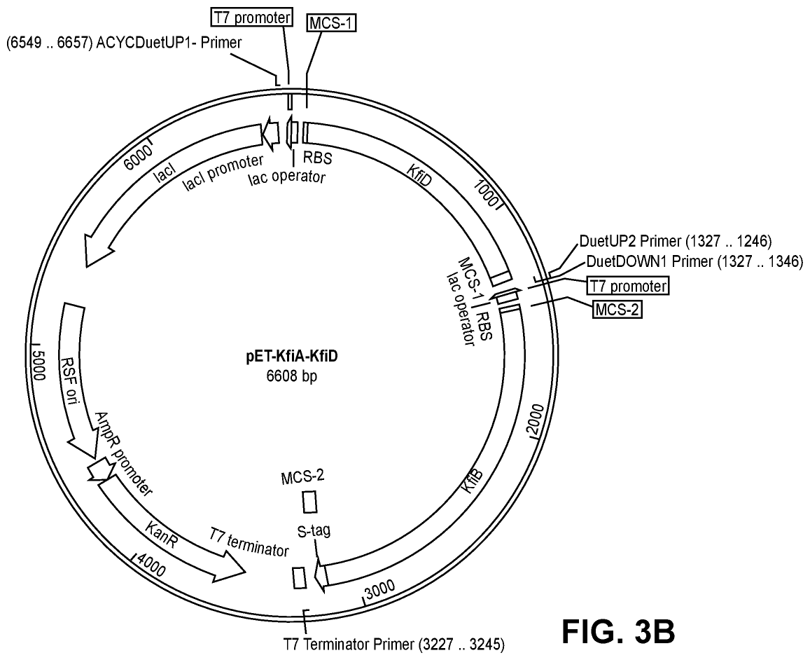

FIGS. 3A and 3B are graphical representations of bacterial plasmid constructs that may be used to prepare E. coli strains BL21-CADB and HT115-CADB.

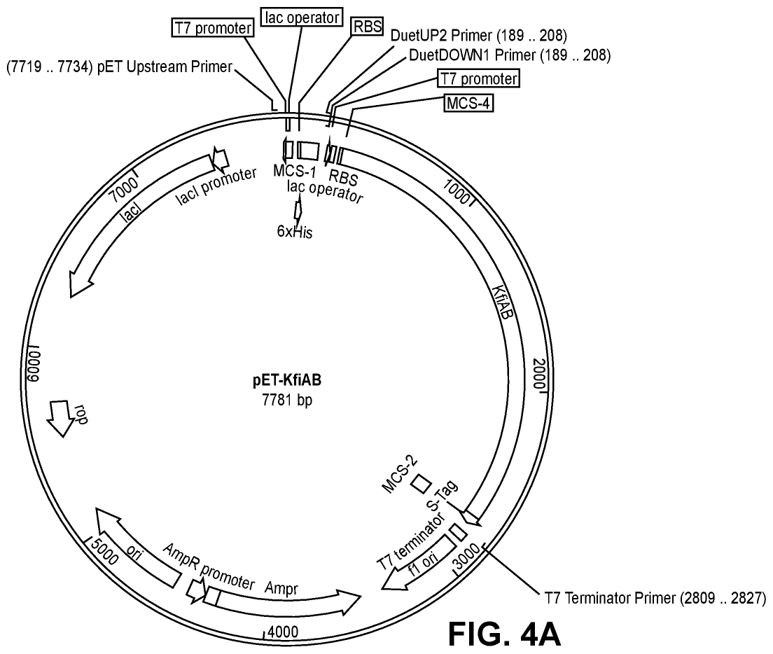

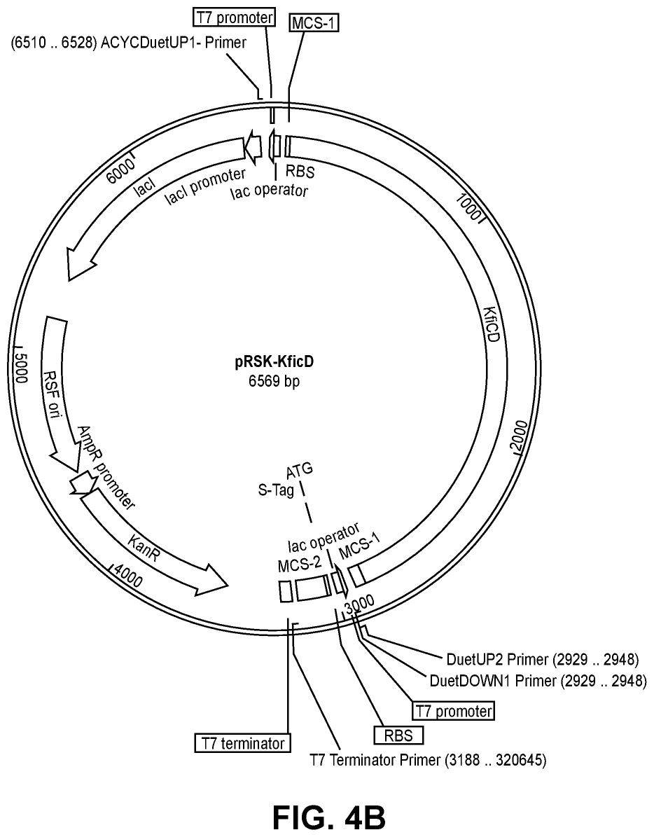

FIGS. 4A and 4B are graphical representations of bacterial plasmid constructs that may be used to prepare E. coli strains BL21-CDAB, HT115-CDAB, and Shuffle-CDAB.

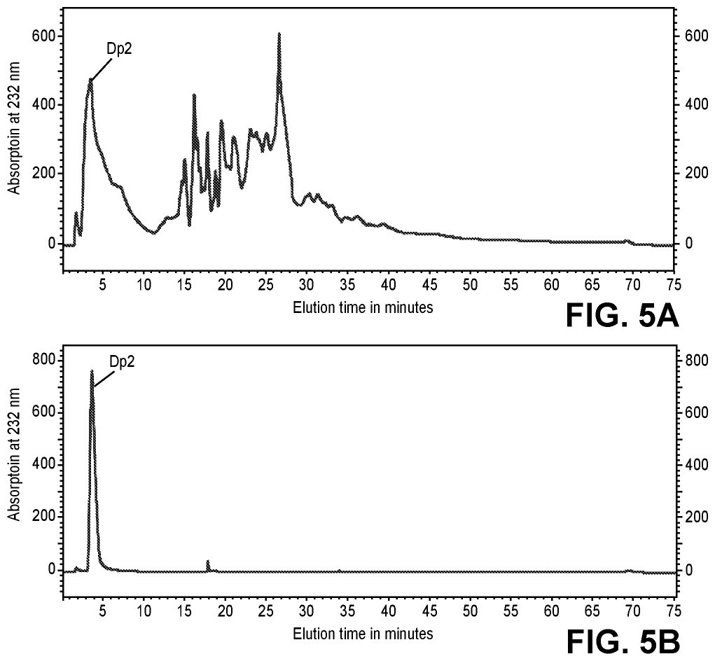

FIGS. 5A and 5B are graphical representations of the purity of recombinant heparosan that may be obtained by culturing the recombinant E. coli strains prepared with the plasmids of FIGS. 3A-4B with different media.

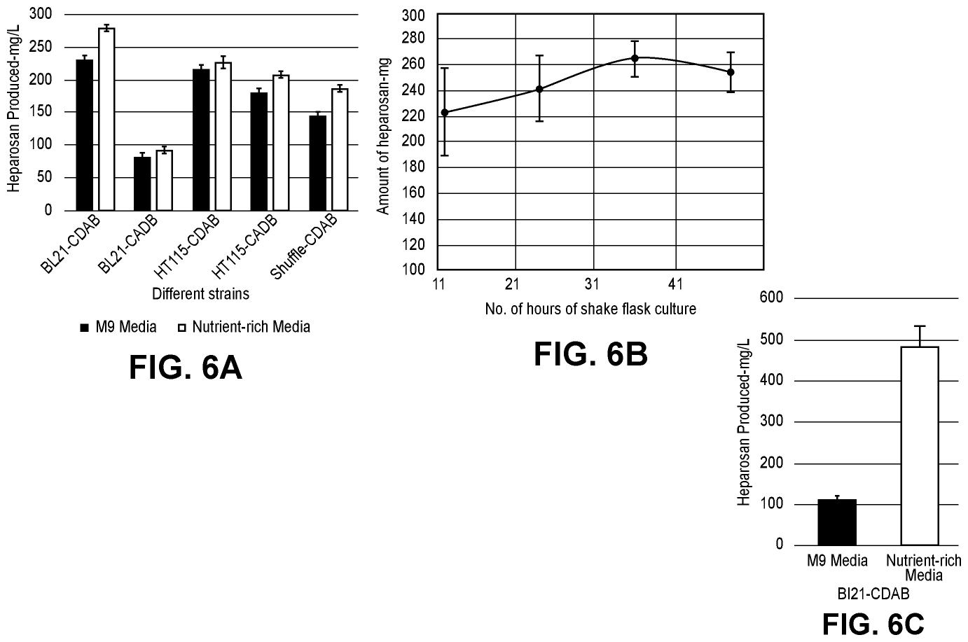

FIG. 6A is a graphical representation of compared heparosan yields from shake flask cultures of the recombinant E. coli strains prepared with the plasmids of FIGS. 3A-4B with different media.

FIG. 6B is a graphical representation of heparosan yields from the shake flask culture at different time points with M9 medium.

FIG. 6C is a graphical representation of heparosan yields from bioreactor cultures of BL21-CDAB strain with different media.

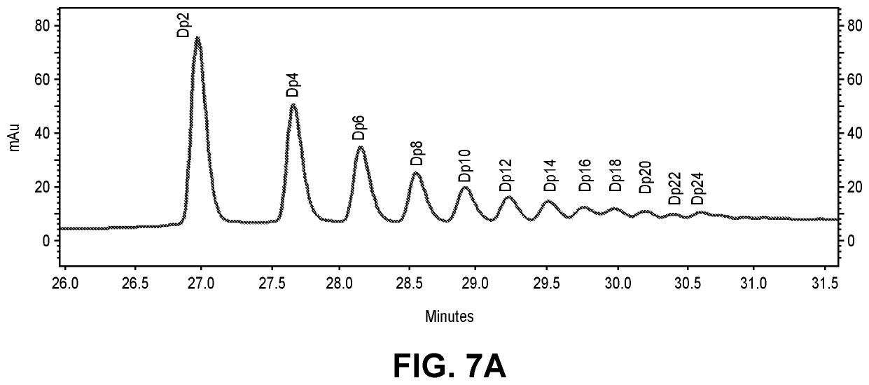

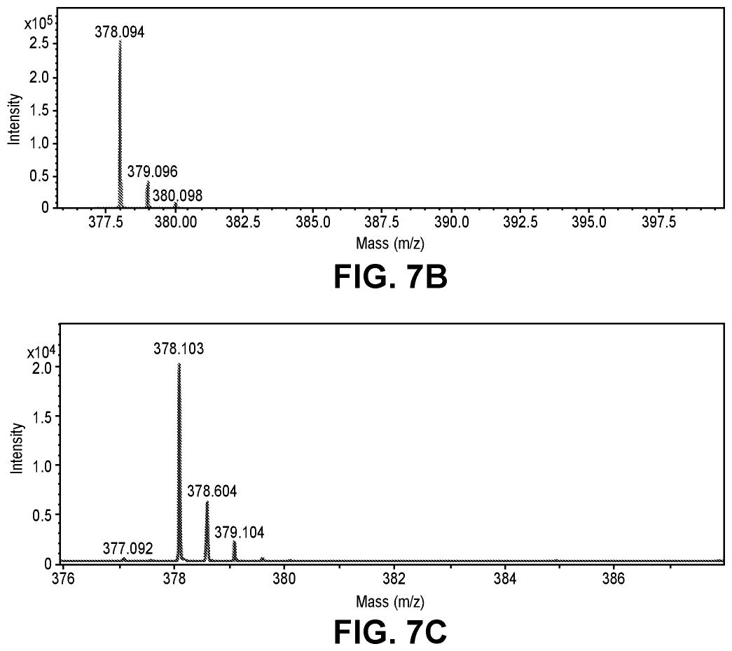

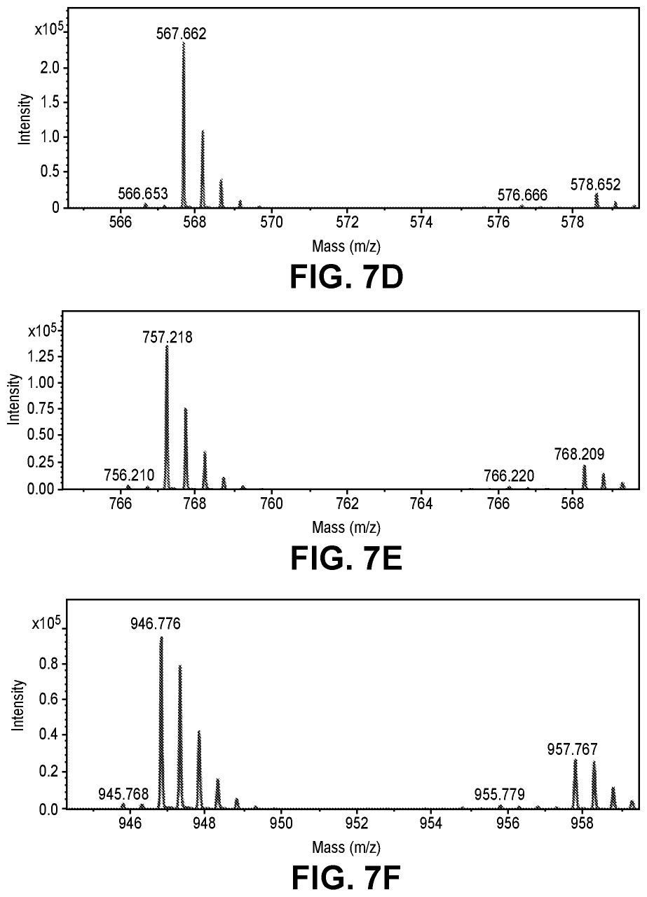

FIG. 7A is a graphical representation of an elution profile of recombinant heparosan oligosaccharides.

FIGS. 7B-7F are electrospray spectra of the heparosan oligosaccharides from FIG. 7A.

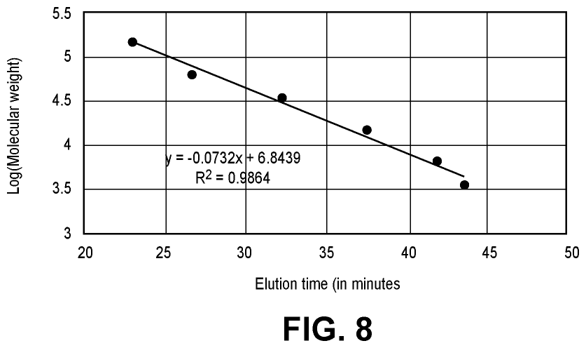

FIG. 8 is a graphical representation of the molecular weights of heparosan polymer using an HPLC size exclusion column, represented by a migration curve.

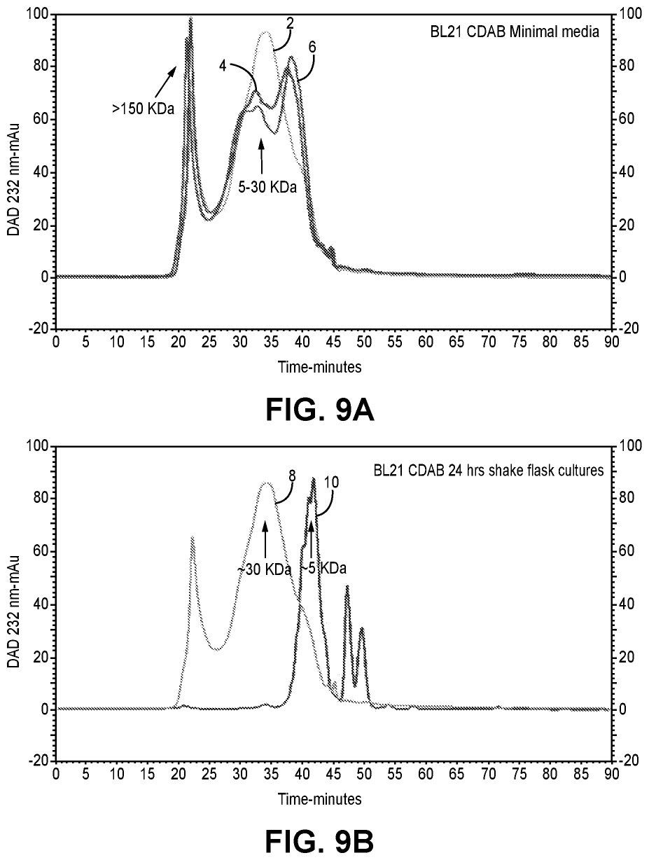

FIG. 9A is a size-exclusion chromatography profile of recombinant heparosan prepared from BL21-CDAB cultures grown in M9 modified culture medium.

FIG. 9B is a size-exclusion chromatography profiles of recombinant heparosan prepared from BL21-CDA13 cultures grown in M9 modified culture medium vs nutrient-rich medium.

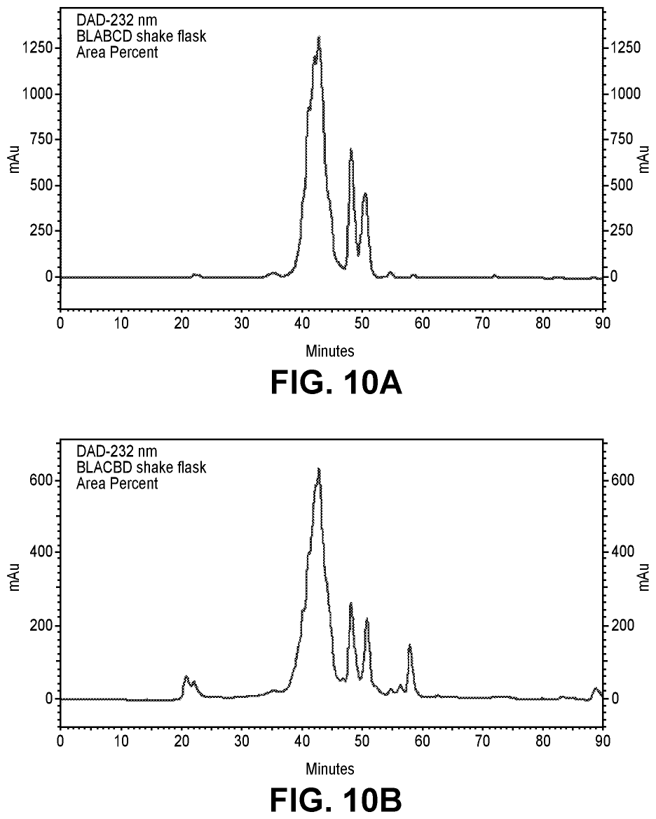

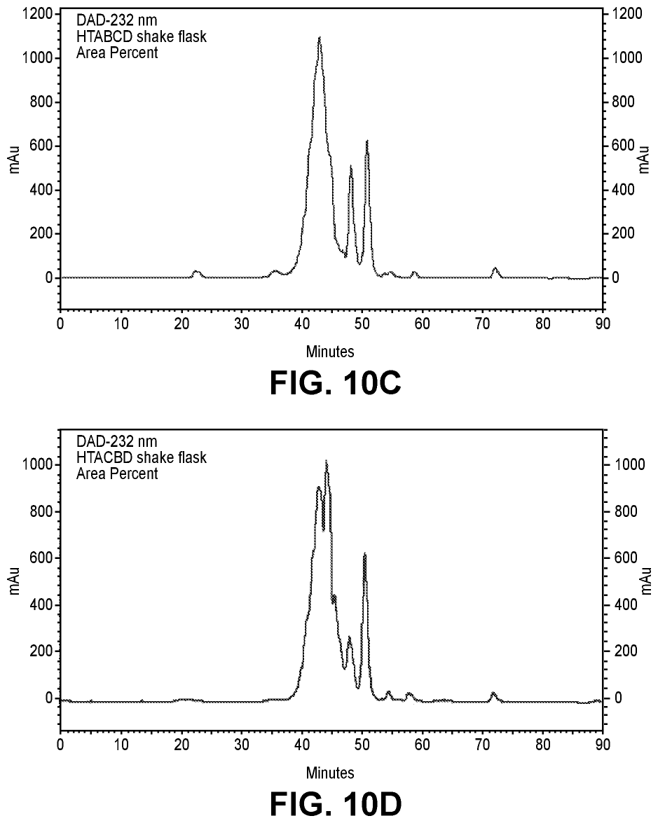

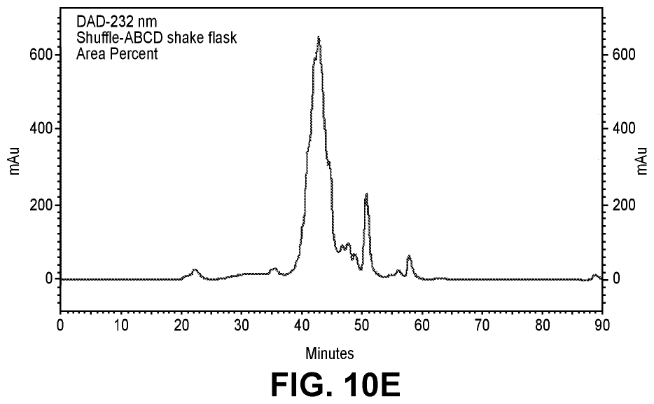

FIGS. 10A-10E are graphical representations of yields of recombinant heparosan produced from shake flask cultures of all the five strains grown for 24 hours in nutrient-rich medium.

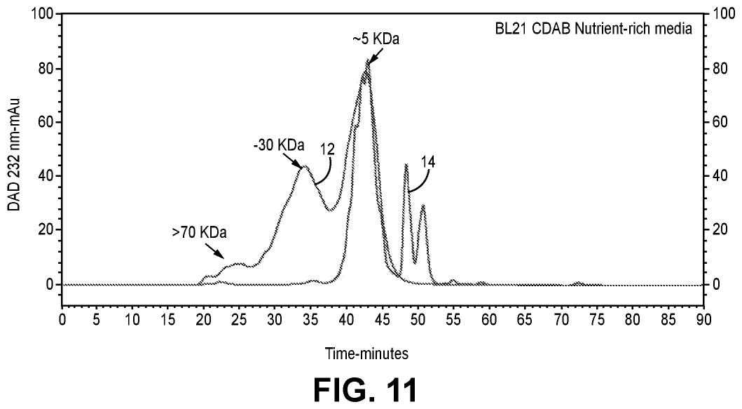

FIG. 11 is a graphical representation of the molecular weights of the isolated heparosan polysaccharide isolated from fed-batch culture.

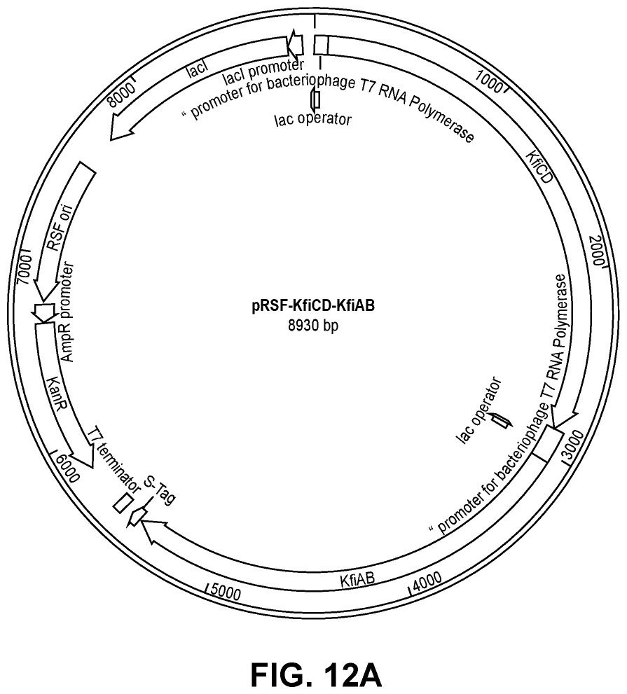

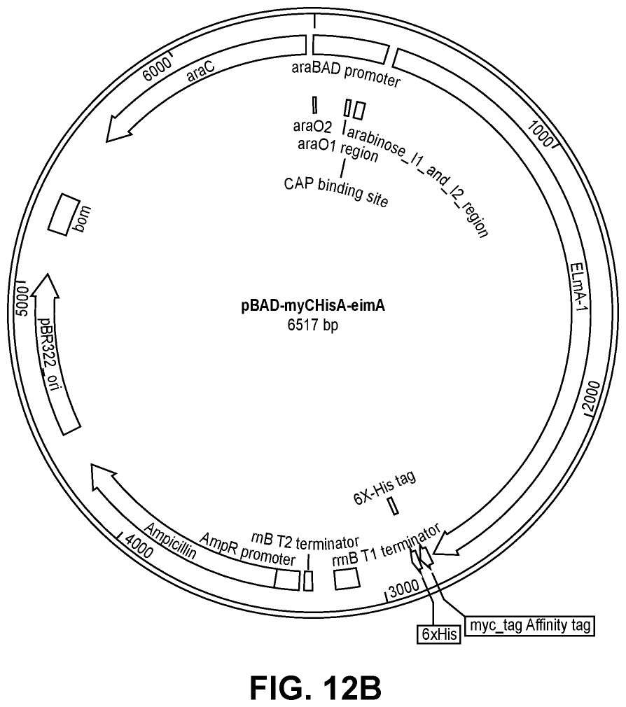

FIG. 12A is a graphical representation of vector pRSF-kfiCD-kfiAB.

FIG. 12B is a graphical representation of vector pRAD-myc/hisA-elmA.

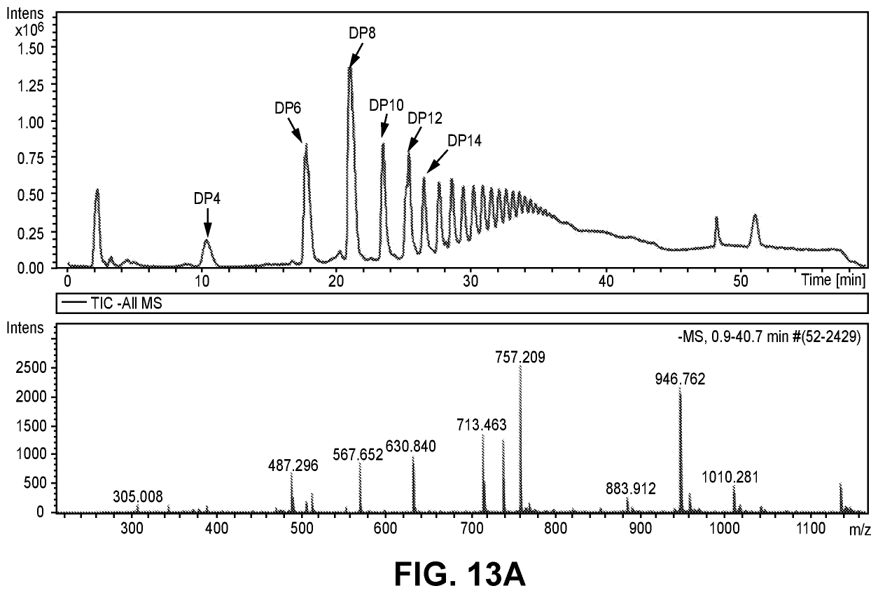

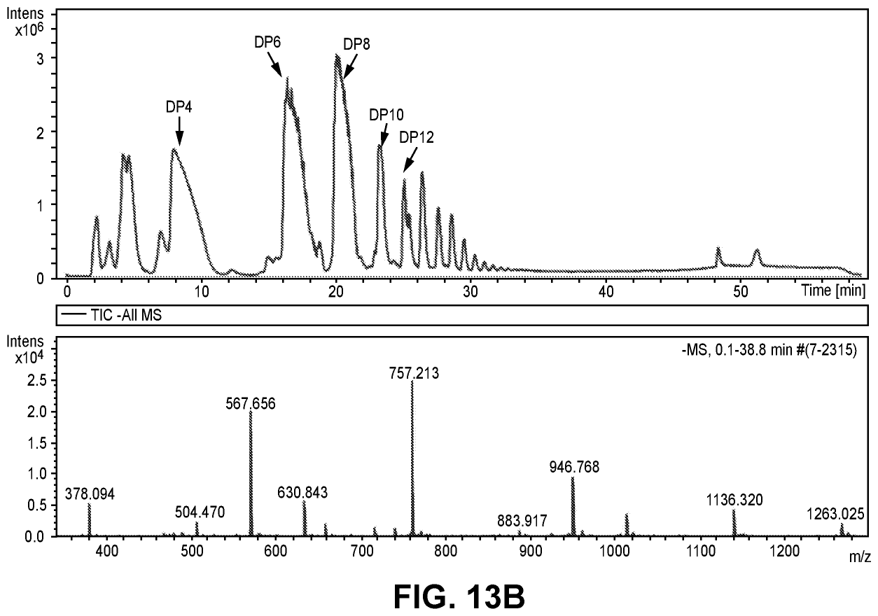

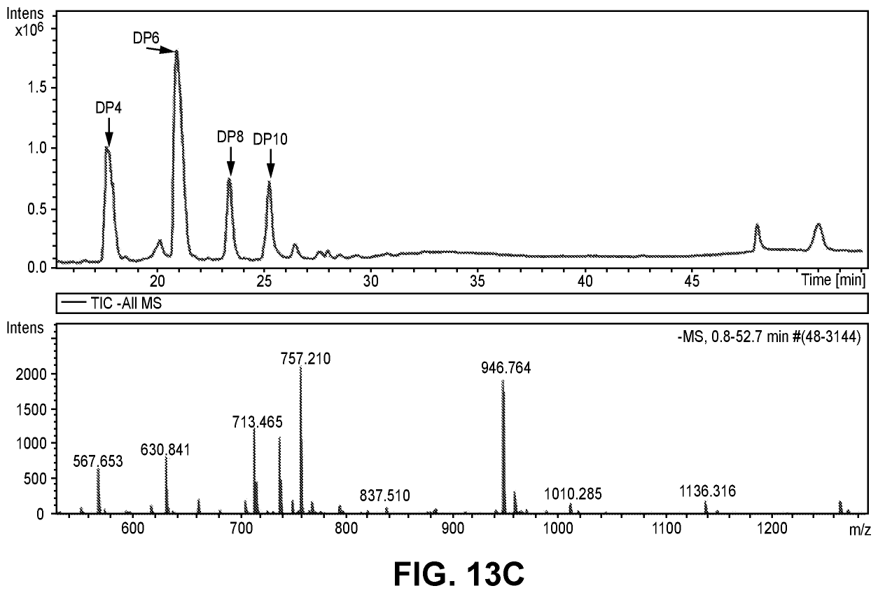

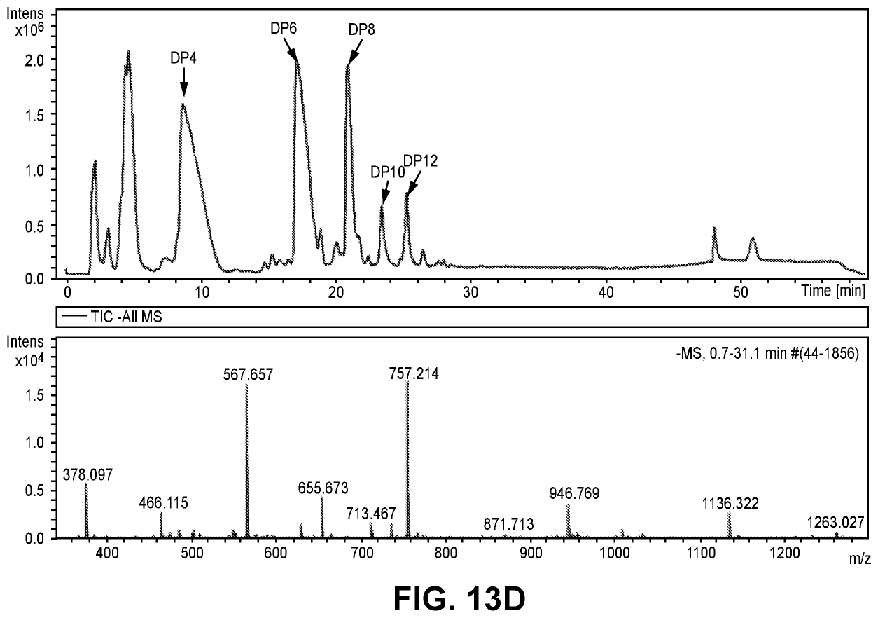

FIGS. 13A-13D are graphical representations of structural analysis of recombinant heparosan oligosaccharides using a capillary HPLC C18 column coupled to an ESI-qTOF-mass spectrometer.

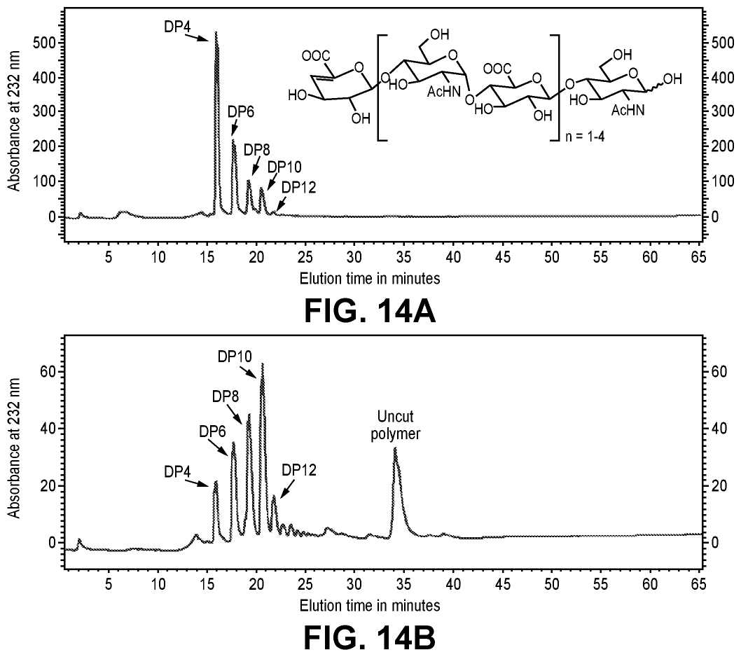

FIGS. 14A and 14B are chromatograms of oligosaccharides obtained after analytical HPLC analysis.

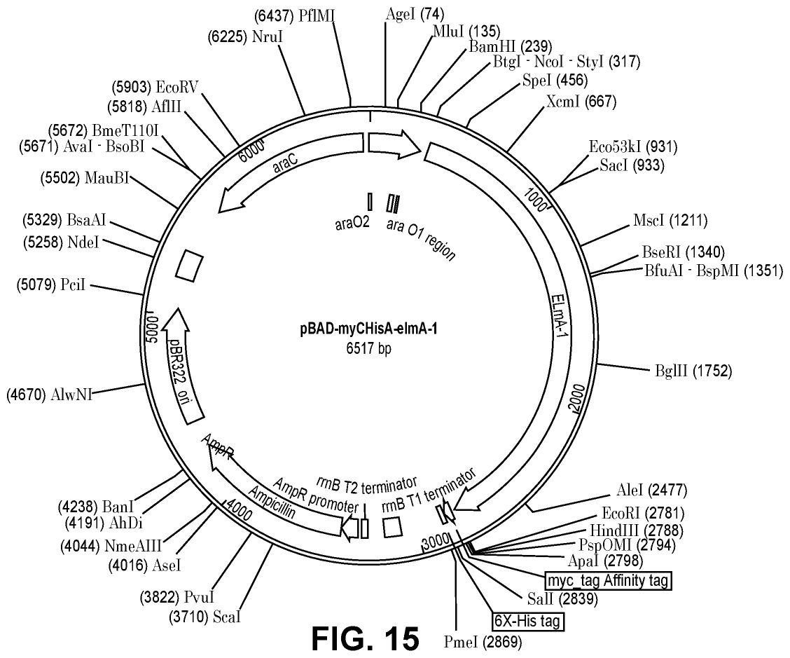

FIG. 15 is a graphical representation of vector pBAD-myc/HisA-elmA-1.

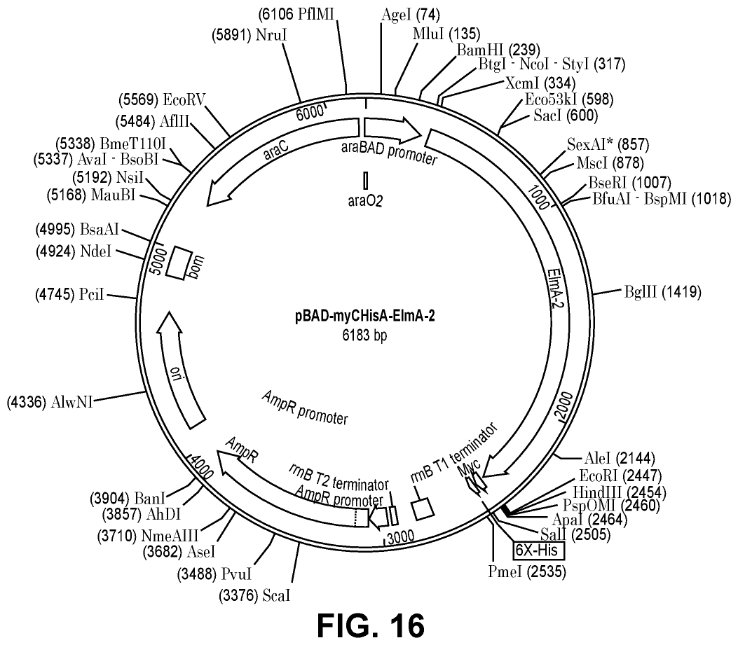

FIG. 16 is a graphical representation of vector pBAD-myc/HisA-elmA-2.

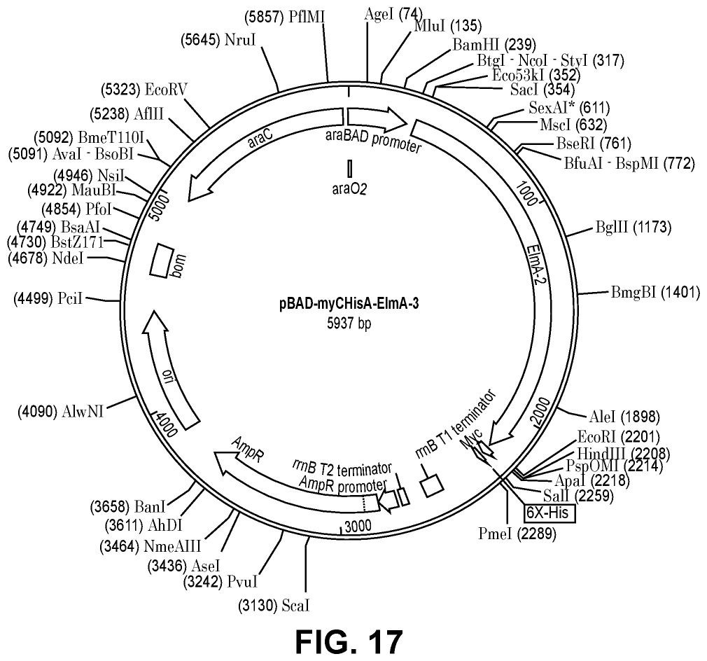

FIG. 17 is a graphical representation of vector pBAD-myc/HisA-elmA-3.

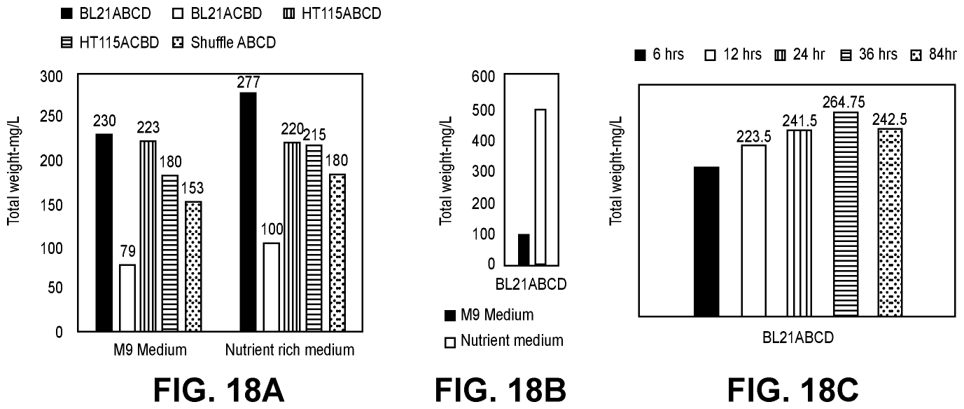

FIG. 18A is a graphical representation of heparosan yields from shake flask cultures of different constructs with different media.

FIG. 18B is a graphical representation of heparosan yields from bioreactor cultures of strain BL21-ABCD with different media.

FIG. 18C is a graphical representation of heparosan yields from shake flask cultures at different time points with M9 medium.

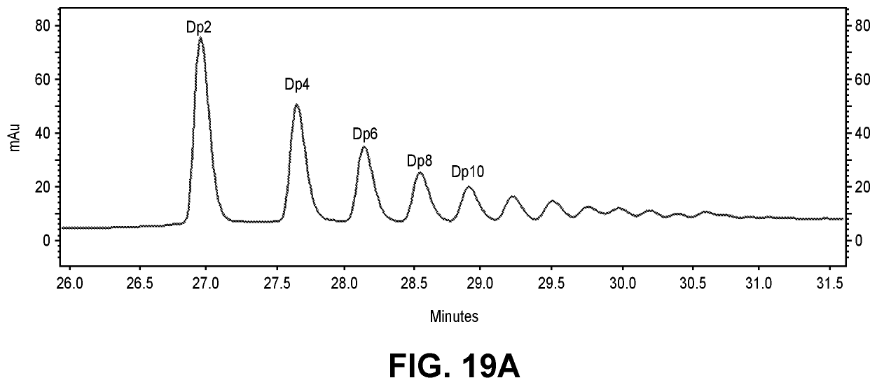

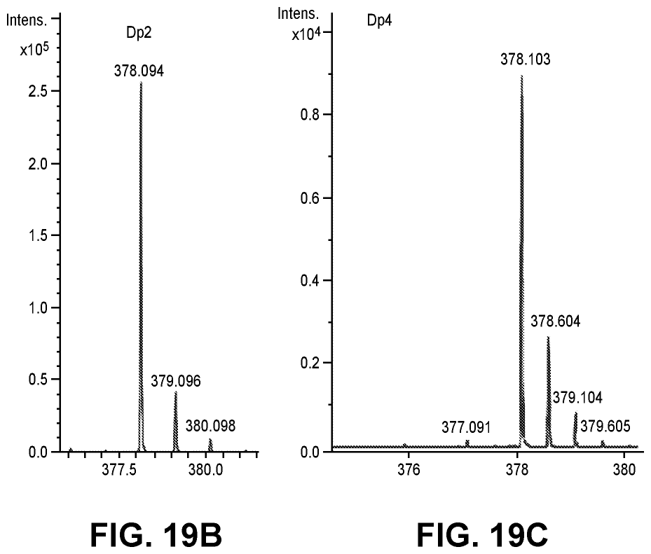

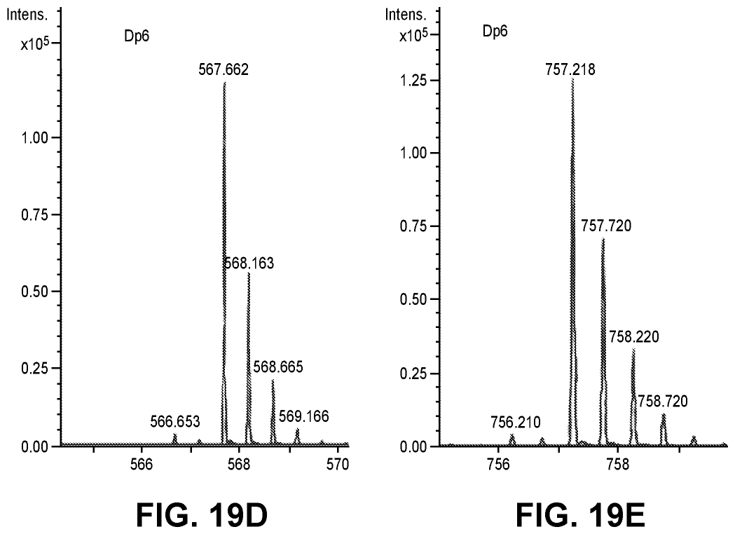

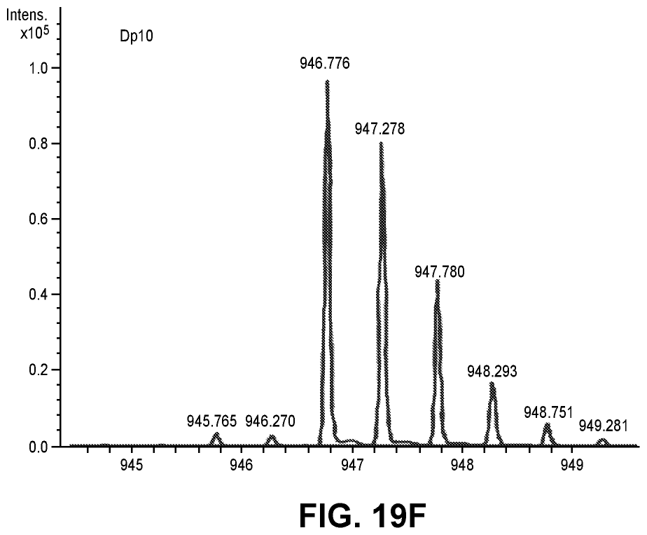

FIGS. 19A-19F are graphical representations of structural analysis of recombinant heparosan oligosaccharides using a capillary HPLC C18 column.

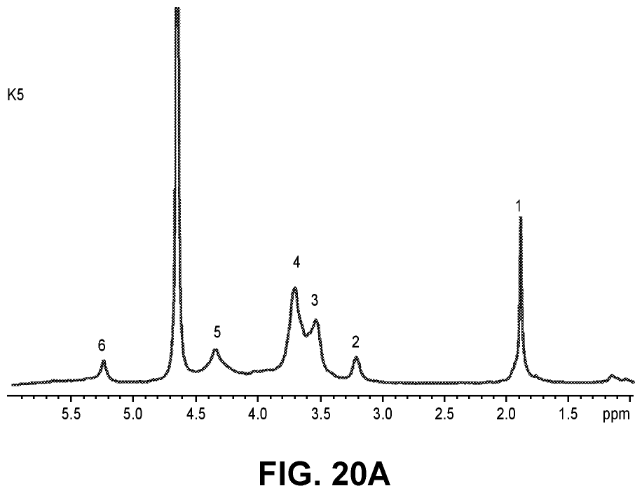

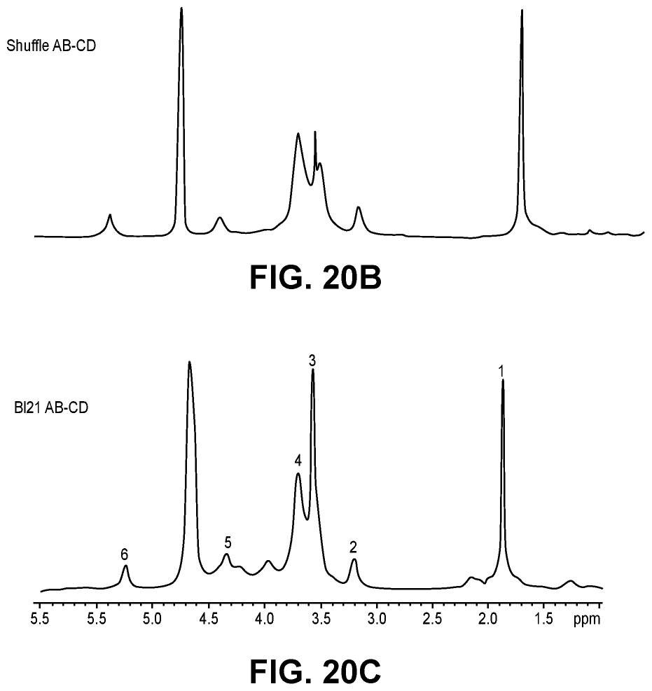

FIGS. 20A-20C are ¹H NMR spectra ref heparosan obtained from E. coli KS, and from recombinant strains E. coli Shuffle T7 B (ShuffleAB-CD) and E. coli BL21 DE3 (BL21 AB-CD).

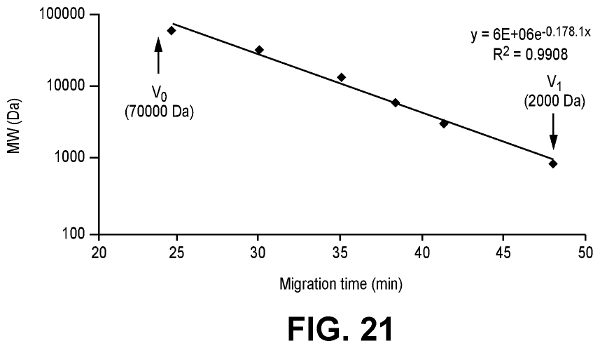

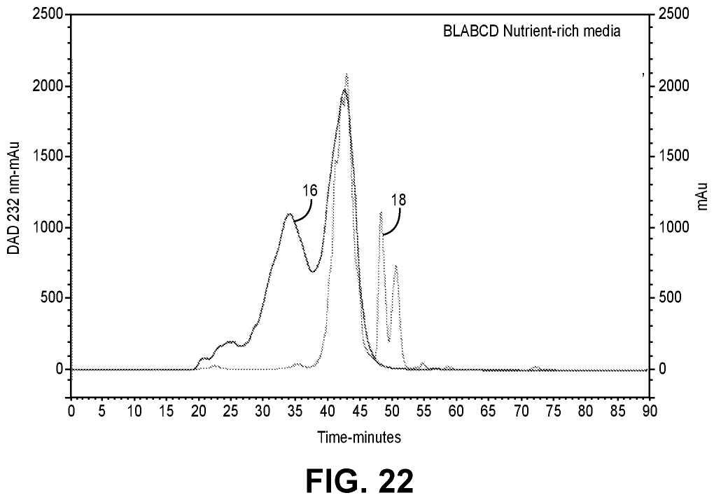

FIGS. 21 and 22 are SEC profiles of K5 and recombinant heparosans.

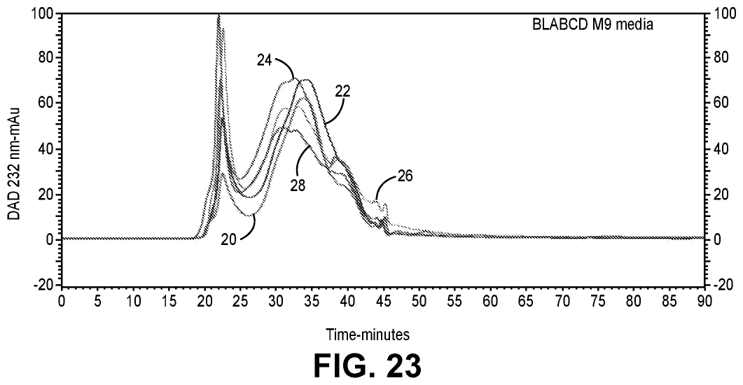

FIG. 23 is an SEC profile of BL21-ABCD culture grown using shake flask and bioreactor methods.

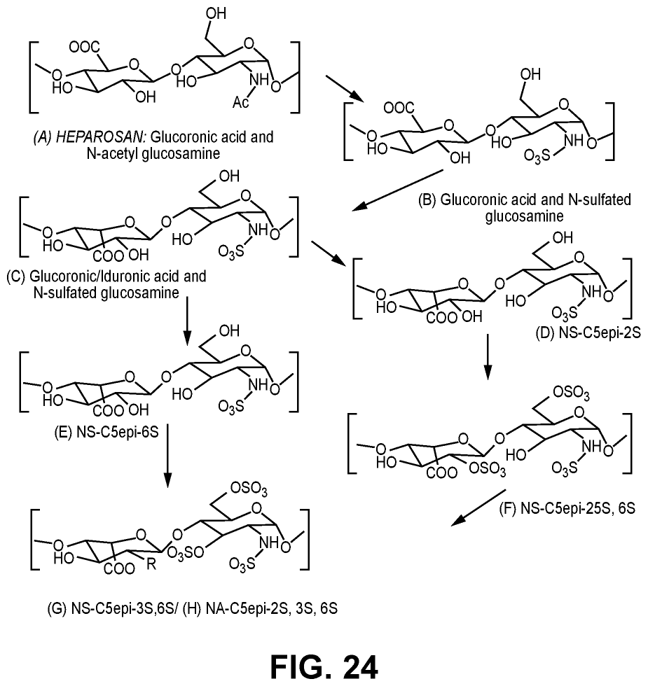

FIG. 24 is a graphical representation of a pathway for the production of heparin-like anticoagulant therapies.

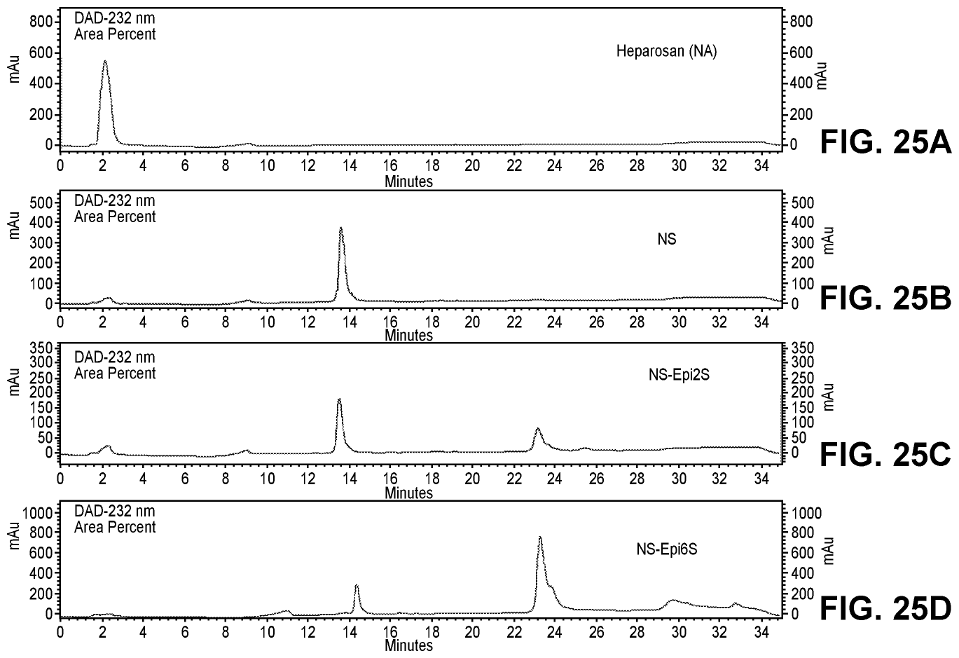

FIGS. 25A-25D are HPLC chromatograms of disaccharides of heparosan before and after various chemical treatments.

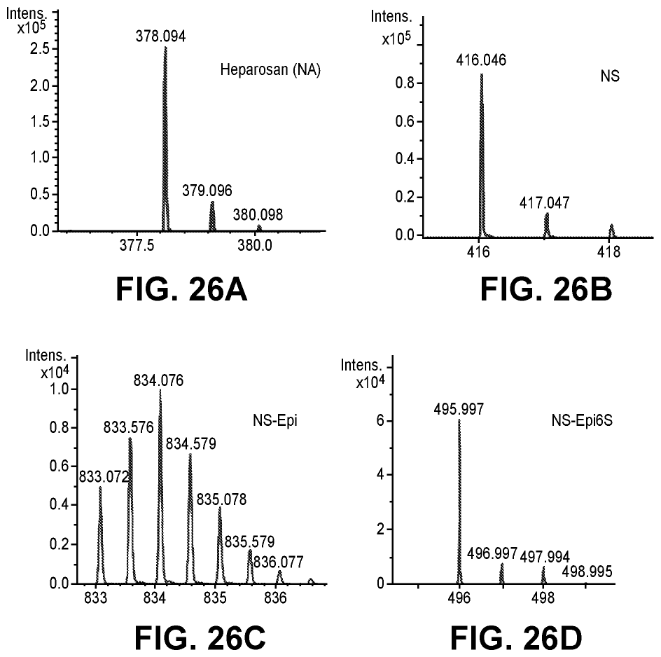

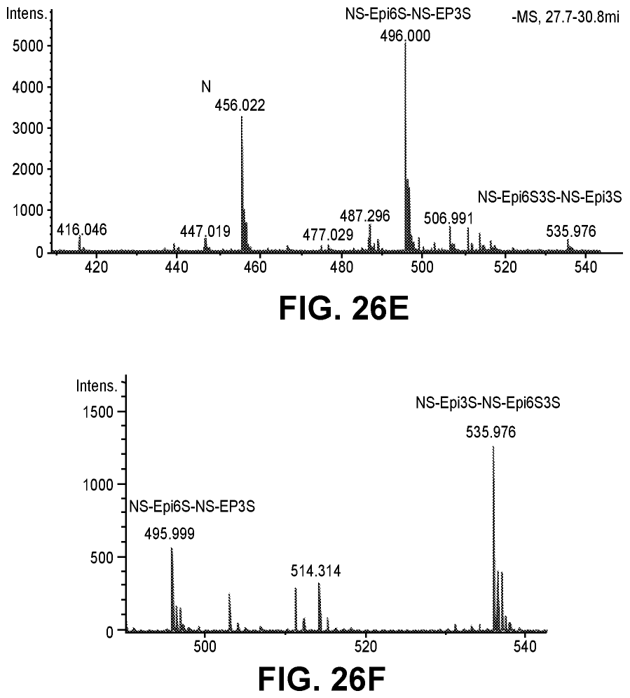

FIGS. 26A-26F are graphical representations of results of LC/MS analysis of heparin-digested polysaccharides.

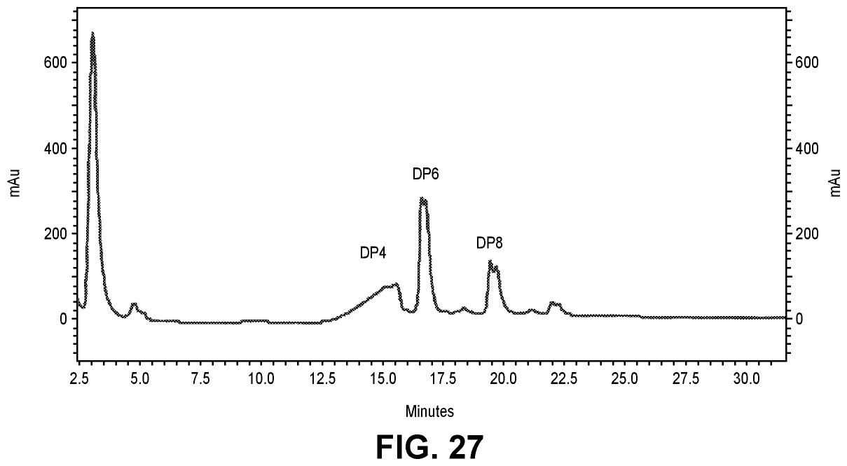

FIG. 27 is an SEC profile of heparosan treated with eliminase for 72 hours.

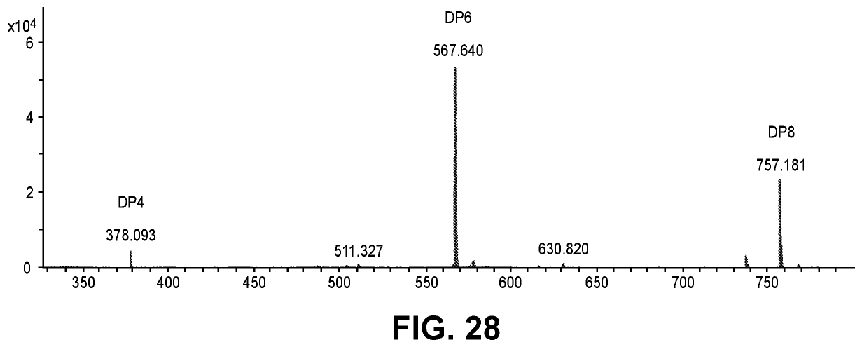

FIG. 28 is a graphical representation of MS analysis of a heparosan sample digested with eliminase A.

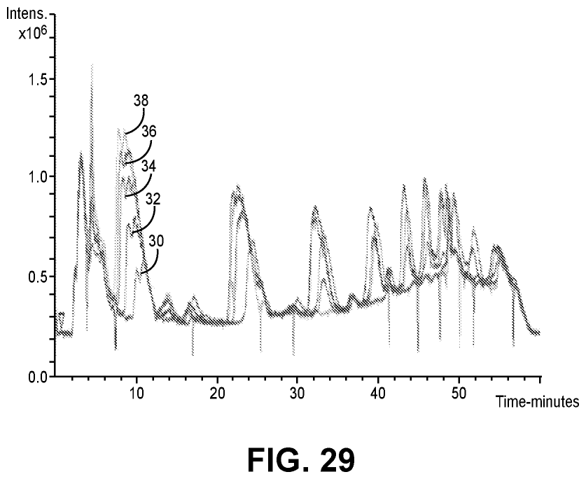

FIG. 29 is a time-dependent digestion profile of oligosaccharides prepared from recombinant eliminase enzyme from the metabolically engineered Top10 E. coli.

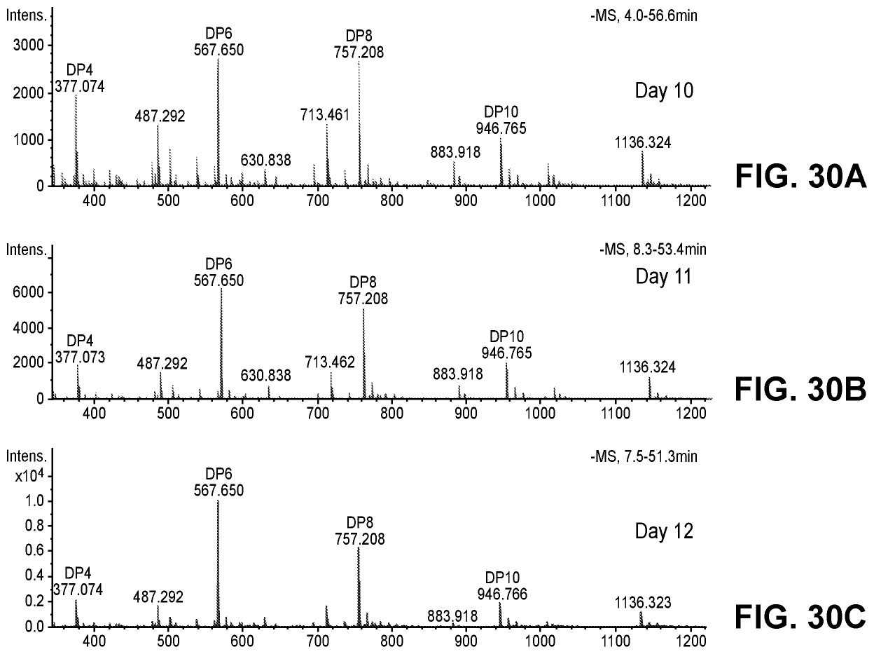

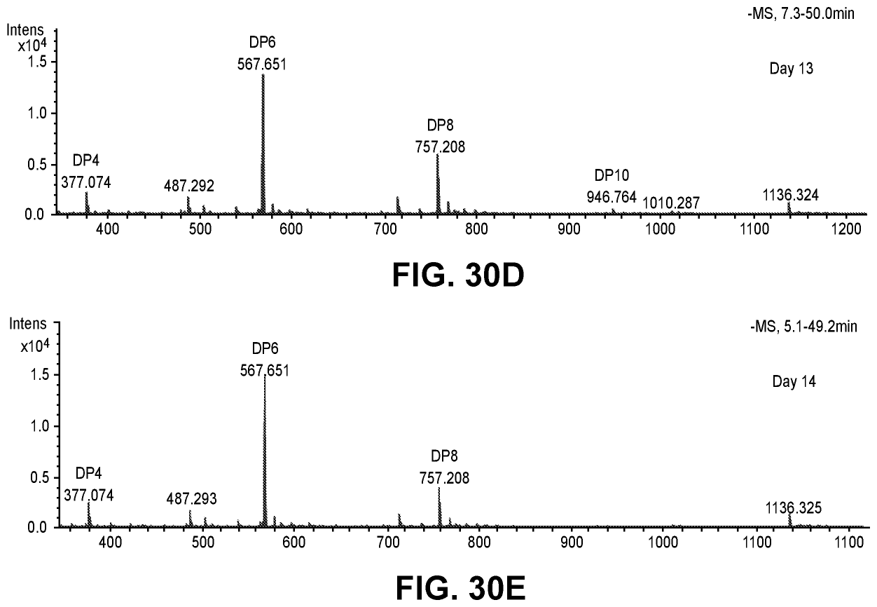

FIGS. 30A-30E are mass spectrometer characterizations of oligosaccharide digestion across a period of time.

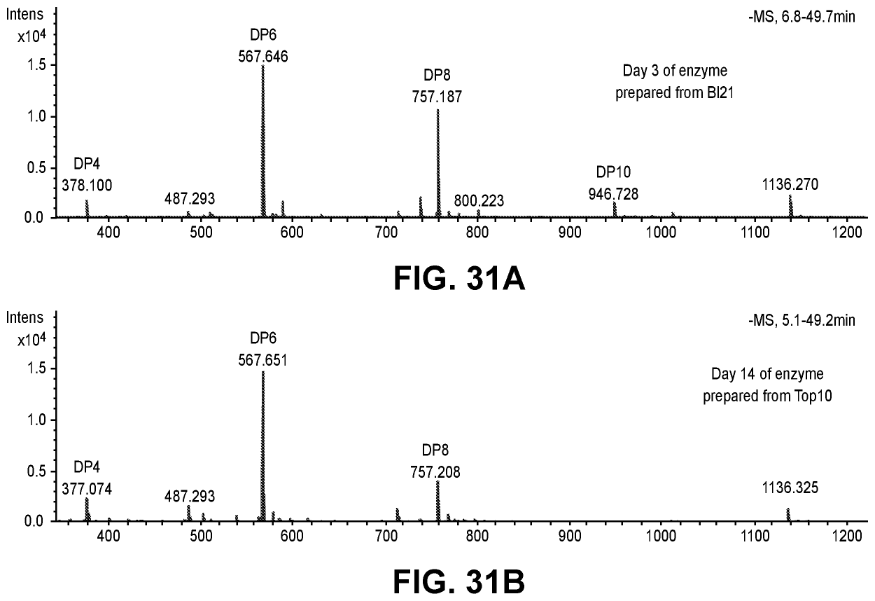

FIGS. 31A and 31B are mass spectrometer characterizations of oligosaccharide profiles after digestion with recombinant Eliminase enzyme.

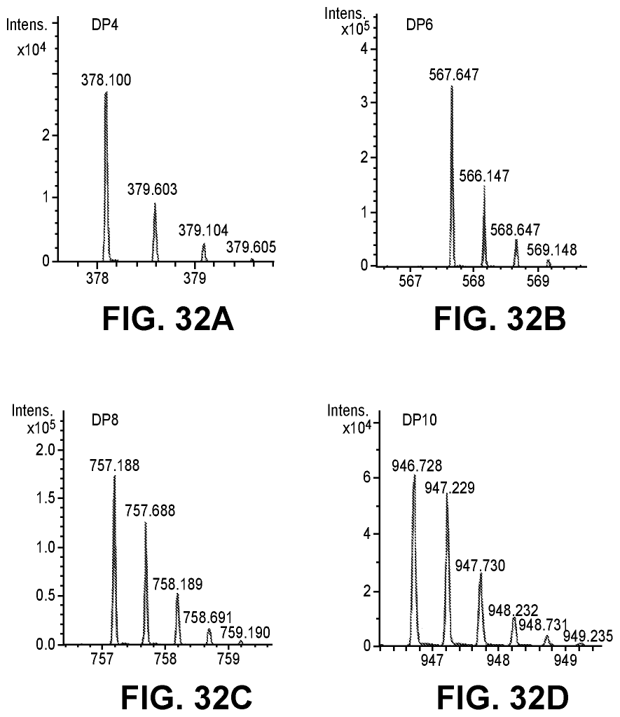

FIGS. 32A-32D are characterizations of tetra-, hexa-, octa-, and deca-saccharides.

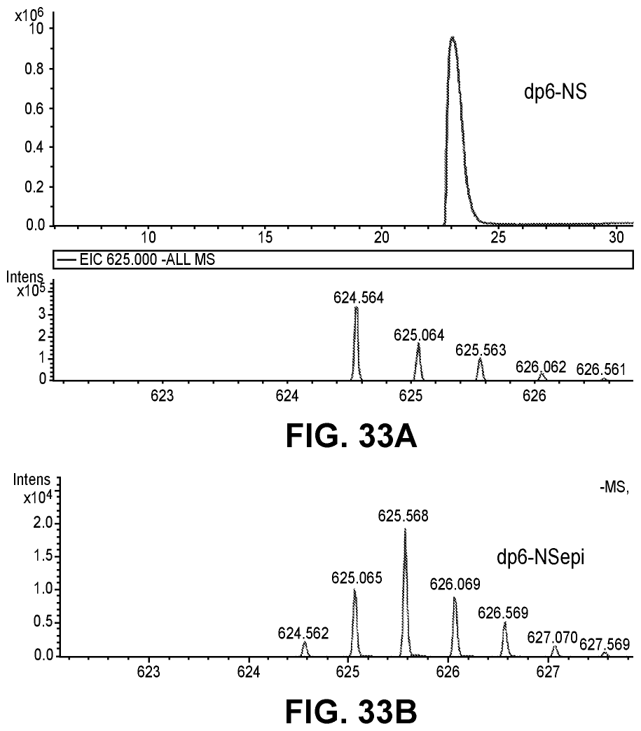

FIGS. 33A and 33B are mass spectrometer characterizations of hexasaccharide after various chemical treatments.

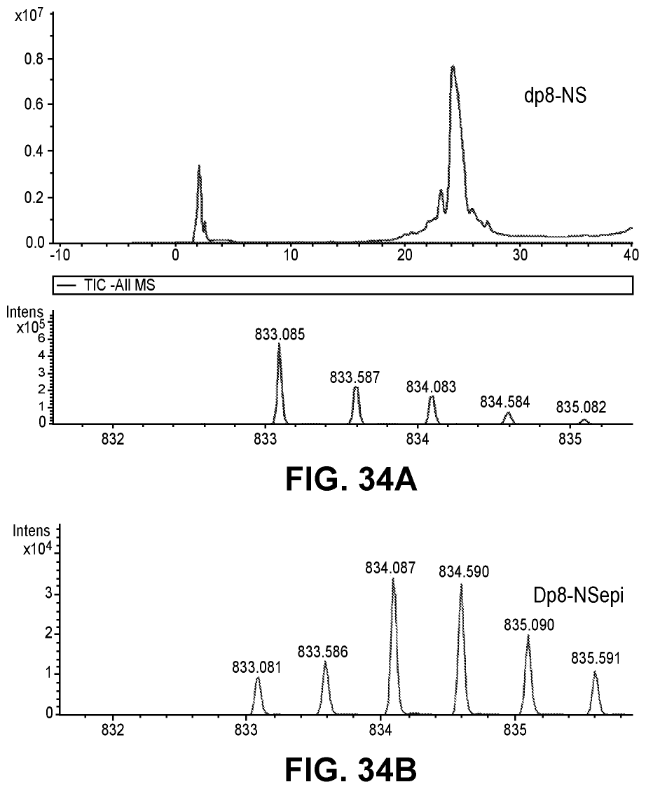

FIGS. 34A and 34B are mass spectrometer characterizations of octasaccharide after various chemical treatments.

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

FIG. 1 is a graphical representation of an overview of the production of heparosan LMWH precursors and heparosan oligosaccharides of specific sizes, as desired, depending on whether the metabolically engineered non-pathogenic strains described herein are co-transformed with an expression vector comprising elmA in addition to the biosynthetic gene cluster KfiA, KfiB, KfiC, KfiD (shown as KfiAB and KfiCD). The metabolically engineered non-pathogenic strains described herein are shown to produce ˜5 kDa heparosan LMWH precursors, for the first time. Additionally, as shown in FIG. 1, heparosan oligosaccharides of specific sizes ranging from tetrasaccharides to dodecasaccharides are directly generated, in a single step, from the recombinant bacterial strains that carry both heparosan biosynthetic genes and an elmA gene.

To arrive at the methods and systems described herein, four essential heparosan biosynthetic genes KfiA (encodes GlcNAc transferase enzyme), KfiB (encodes a polymerase factor), KfiC (encodes GlcA transferase enzyme) and KfiD (encodes UDP-Glucose dehydrogenase enzyme) were cloned in different orders within several expression vectors and transformed into three non-pathogenic E. coli strains [i.e., BL21(DE3), HT115(DE3), Shuffle T7 Express B] to create several distinct recombinant strains. By applying different cloning orders, host organisms, culture medium compositions, and length of culture times, the chain length of heparosan produced may be controlled, ranging from high molecular weight heparosan (>150 kDa) to low molecular weight heparosan (˜5 KDa).

As shown in FIG. 1, the elmA gene, found in E. coli K5, which encodes the eliminase enzyme that cleaves heparosan polysaccharide into smaller oligosaccharides, may be cloned. An earlier study has shown that induction of both biosynthetic gene cluster and elmA together can generate various oligosaccharide sizes without an ability to control the range of oligosaccharides produced. This disclosure describes an approach, through a combined expression of the heparosan biosynthetic and eliminase gene products under two different induction systems, to control the chain length of heparosan oligosaccharides generated from the metabolically engineered non-pathogenic strains, ranging from tetrasaccharides to dodecasaccharides, in a single step.

Production of low-molecular and high-molecular-weight heparosan polymers. FIG. 2 illustrates the cloning strategy of the different combinations of KfiABCD gene constructs and bacterial strains for heparosan production. As shown, the genes required for heparosan biosynthesis may be cloned from the region 2 (KfiABCD, FIG. 2) of the heparosan biosynthetic gene cluster of E. coli K5. It is highly desirable to control the recombinant heparosan polymer chain length while improving the yield. One factor associated with controlling the production of recombinant heparosan of various molecular weights is the cloning order of the genes in different host organisms. To achieve this, the genes were initially cloned as described herein.

FIGS. 3A and 3B illustrate bacterial plasmid constructs that may be used to prepare E. coli strains BL21-CADB and HT115-CADB. KfiA and KfiC may be cloned separately into multiple-cloning sites (MCS) 1 and 2, respectively, of the plasmid pET-Duet1 to prepare construct pET-KfiC-KfiA and the genes KfiB and KfiD may, similarly, be cloned separately into different MCS of the plasmid pRSF-Duet1 to prepare construct pRSF-KfiD-KfiB (FIGS. 3A and 3B). Constructs pET-KfiC-KfiA, pRSF-KfiD-KfiB then may be co-transformed into E. coli BL21(DE3) and HT115(DE3) to create engineered strains BL21-CADB and HT115-CADB, respectively (FIG. 2).

FIGS. 4A and 4B illustrate the bacterial plasmid constructs pET-KfiAB and pRSF-KfiCD, which may be used to prepare E. coli strains BL21-CDAB, HT115-CDAB, and Shuffle-CRAB. Multicistronic units (endogenous ribosome entry sites) may naturally exist in the intronic regions between genes KfiA and KfiB, and KfiC and KfiD. Therefore, in one embodiment, the entire region, from gene KfiA to gene KfiB, may be cloned into the plasmid pET-Duet1 to create construct pET-KfiAB (FIG. 4A). Similarly, the complete region, from gene IOC to gene KfiD, may be cloned into the plasmid pRSF-Duet1 to create construct pRSF-KfiCD (FIG. 4B). The constructs, pET-KfiAB and pRSF-KfiCD, then may be co-transformed into the E. coli strains BL21(DE3), HT115(DE3) and Shuffle T7 B (DE3) to create recombinant strains BL21-CDAB, HT 115-CDAB and Shuffle-CDAB, respectively (FIG. 2). In this manner, five recombinant E. coli strains have been engineered with genes cloned in different orders into expression vectors to investigate their potential impact on the production of heparosan in terms of molecular weight and yield.

FIGS. 5A and 5B illustrate the purity of recombinant heparosan that may be obtained by culturing recombinant bacteria with different media. Culture conditions, particularly the medium composition and culture duration time, are additional factors affecting recombinant heparosan yield and molecular weight. The embodiment of FIG. 5A illustrates an outcome from recombinant bacteria grown in media containing partially-hydrolyzed protein sources (tryptone or peptone; e.g., Terrific Broth medium, available from Sigma-Aldrich) for various lengths of time. In this embodiment, the resulting heparosan samples were found to retain a significant amount of protein impurities even after extensive dialysis and protease treatment, as shown in FIG. 5A.

In another embodiment, the protein-free M9 minimal medium may be inoculated as an overnight culture of the recombinant bacteria grown in LB medium in the ratio of 1:10 v/v (LB: M9). From the culture of this embodiment, recombinant heparosan without protein impurities may be prepared relatively easily, as shown in FIG. 5B.

FIG. 6A illustrates compared heparosan yields from shake flask cultures of the five recombinant E. coli strains described above with different media. In an evaluation of the five recombinant E. coli strains described above, the yields of heparosan obtained after 24 hours of shake flask cultures from all the five strains were compared, indicating that the highest heparosan yield (˜230 mg/L) of such recombinant strains may be achieved with the BL21-CDAB strain, as shown in FIG. 6A. FIG. 6B illustrates heparosan yields from the shake flask culture of at different time points with M9 medium. An investigation of optimal culture time durations indicates that highest heparosan yield may be obtained around -36 hours, as shown in FIG. 6B. FIG. 6C illustrates heparosan yields from bioreactor cultures of BL21-CRAB strain with different media. In the embodiment of FIG. 6C, recombinant heparosan was prepared on a larger scale using a 6 L batch fermenter from BL21-CDAB strain grown in M9 medium for ˜40 hours after induction. Fed-batch fermenter cultures may provide a relatively lower yield in comparison to those obtained from shake flask cultures. As shown in FIG. 6C, these cultures gave, in one example, an average yield of 113 mg/L heparosan, significantly less than the ˜230 mg/L yields obtained from the shake-flask culture examples of FIGS. 6A and 6B.

Following culture, structural analysis may be carried out to confirm the heparosan fine structure. FIG. 7A illustrates an example analysis of an elution profile of recombinant heparosan oligosaccharides obtained by partial enzymatic digestion of heparosan polymer with heparinase enzyme using an analytical HPLC Carbopac anion-exchange column (available from Dionex). With respect to the engineered non-pathogenic E. coli strains described above, HPLC analysis revealed that disaccharide composition of the recombinant heparosan, isolated from all said non-pathogenic strains, was similar to that of heparosan derived from the pathogenic E. coli K5 strain. In the illustrated example, partial digestion of recombinant heparosan from E. coli BL21-CDAB with heparinase I enzyme for 5 minutes gave a chromatographically well-resolved oligosaccharide profile, ranging from DP2 to DP24, as shown in FIG. 7A. The well-isolated peaks resulting from this analysis (e.g., from FIG. 7A) may he collected with the aid of fraction collector and further analyzed using a capillary HPLC C18 coupled to ESI-qTOF mass spectrometer to confirm the expected molecular weight of each oligosaccharide, as illustrated by example in FIGS. 7B-7F. FIGS. 7B-7F are electrospray spectra of the heparosan oligosaccharides from FIG. 7A, ranging in size from disaccharide (DP2) to decasaccharide (DP10). FIG. 7B shows (M-1H)⁻¹ profile of DP2 whereas FIGS. 7C-7F respectively show (M-2H)⁻² profiles of DP4, DP6, DP8, and DP10.

Next, the 1H-NMR analyses of heparosan polysaccharide isolated from E. coli K5 and recombinant E. coli BL21(DE3) strains (BL21-CDAB and BL21-CADB), E. coli HT115 DE3 (HT115-CDAB and HT115-CADB), and E. coli Shuffle T7 Express B (Shuffle-CDAB) may be performed to confirm the heparosan structure. Finally, the molecular weights of heparosan polymer may be analyzed using an HPLC size exclusion column by comparing their elution times with migration times observed for polysulfonated standards, as illustrated in FIG. 8. FIG. 9A is the size-exclusion chromatography profile of recombinant heparosan prepared from BL21-CDAB cultures grown in M9 modified culture medium at 24 hours in shake flask culture (line 2), 36 hours in shake flask culture (line 4), and at 48 hours in shake flask culture (line 6). The BL21-CDAB cultures grown in M9 modified culture medium gave one significant product size of >150 kDa. (ultra-high molecular weights) and a mixture of product sizes ranging between 5-30 kDa, as illustrated in FIG. 9A.

Embodiments of the present disclosure further include methods for improving yields of recombinant heparosan produced on a larger scale using a 6 L batch fermenter. The low yields that may in some instances be obtained from the use of a 6 L batch fermenter, as described above, may be attributable to insufficient carbon and protein sources in the medium. To improve yields of low molecular weight heparosan when using a 6 L batch fermenter, a nutrient rich medium with casamino acids (as a protein source) and glycerol (as a carbon source) may he used as further described below under Media Conditions, In an example trial, such nutrient-rich media substantially improved the yield compared to minimal medium in shake flask cultures (grown for 24 hours at 37° C.) of all the strains, E. coli BL21-CDAB, HT115-CDAB, BL21-CADB, HT115-CADB, and Shuffle-CDAB. (FIG. 6A), while the amount of impurities was negligible. Importantly, in this trial, the recombinant heparosan produced from shake flask cultures of all the five strains grown for 24 hours in nutrient-rich medium consistently gave low molecular weight heparosan of 5 KDa or below (FIGS. 9B and FIGS. 10-10E). FIG. 9B shows the molecular weights of heparosan obtained from the BL21-CDAB strain cultured for 24 hours in shake flasks containing minimal medium (line 8) or nutrient-rich medium (line 10). Since the highest heparosan yield in this trial (˜280 mg/L) was obtained from the BL21-CRAB strain, in a nutrient-rich medium, under shake flask culture conditions (FIG. 6A), this same strain and medium may be used for hatch bioreactor cultures to achieve similar yields. To further improve heparosan cultures may be grown in a 6 L-bioreactor, under fed-batch conditions, with nutrient-rich buffer containing 50% glycerol added at 1.8 mL/hr. Once OD₆₀₀ of 18.0 is reached, the heparosan expression may be induced and followed with ˜40 hours of culture. In trials, these cultures produced recombinant heparosan with a final yield of ˜480 mg/L after purification (FIG. 6C). The molecular weight of the isolated heparosan polysaccharide isolated from the fed-batch culture was a mixture of two major sizes: ˜5 KDa and ˜30 KDa (FIG. 11 ). FIG. 11 shows the molecular weights of heparosan obtained from the BL21-CDAB strain cultured in nutrient-rich media for 48 hours in batch culture supplemented with glycerol in a bioreactor (line 12) or in shake flask culture for 24 hours (line 14).

Production of size-specific oligosaccharides. As discussed above, the minimum size of heparin, which is required for binding to antithrombin III and to function as an anticoagulant, is a pentasaccharide. Additionally, production of size- and sulfation-specific oligosaccharides are required for the study of the structure-function relationship of heparin-like structures with various proteins. Conventional approaches of preparing size-specific oligosaccharides from polysaccharides, through partial digestion using recombinant heparin lyases, are inefficient as heparin lyases cleave the polymer through processive exolytic mechanism resulting predominantly in the generation of disaccharides. To overcome these issues with conventional approaches, the elmA gene, from the pathogenic E. coli strain K5, which specifically generates larger oligosaccharides may be used in engineered bacterial strains as discussed herein.

To directly produce heparosan oligosaccharides using engineered bacterial strains, elmA may be cloned into the arabinose-inducible pBADmyc/hisA expression vector (pBAD-myc/hisA-elmA, arabinose inducible). Additionally, KfiAB may be cloned into the MCS2 site and KfiCD into the MCS1 site of pRSF-Duet1 (lac operon system) to prepare an expression vector, pRSF-kfiCD-kfiAB. The two vectors, pRSF-kfiCD-kfiAB (FIG. 1.2A) and pBAD-myc/hisA-elmA (FIG. 12B), may be co-transformed into E. coli BL21(DE3) to create the strain BL21-CDAR-elmA. IPTG may be added for the production of heparosan using pRSF-kfiCD-kfiAB and arabinose for the expression of the eliminase enzyme using pBAD-myc/hisA-elmA. Thus, utilizing two different expression systems allowed for the induction of heparosan polysaccharide synthesis and eliminase activities, either sequentially or concurrently.

In addition, depending on differences in induction (concurrent or sequential), culture temperature (30° C. and 37° C.) and duration of culture (24 or 48 hours), the composition of oligosaccharide sizes synthesized by the bacterial cultures may be readily controlled as shown in FIGS. 13A-13D, which illustrate structural analysis of recombinant heparosan oligosaccharides using a capillary HPLC C18 column coupled to an ESI-qTOF-mass spectrometer. In the example of FIGS. 13A-13D, the recombinant heparosan oligosaccharides, which were obtained from cultures of the strain BL21-CDAB-elmA. The culture of FIG. 13A was induced with IPTG at OD=˜0.5 and cultured for 24 hrs at 37° C., followed by induction with Arabinose for 24 hrs at 30° C., The culture of FIG. 13B was induced with IPTG at OD=˜0.5 and cultured for 24 hrs at 30° C. followed by induction with Arabinose for 24 hrs at 30° C. The culture of FIG. 13C was induced with IPTG and Arabinose together at OD=˜0.5 and cultured for 24 hrs at 30° C. The culture of FIG. 13D induced with IPTG and Arabinose together at OD=−0.5 and cultured for 24 hrs at 37° C.

FIGS. 14A and 14B are chromatograms of oligosaccharides obtained after analytical HPLC analysis. FIG. 14A illustrates mostly tetrasaccharide (DP4) as a major peak obtained a from BL21-CDAB-elmA culture when induced with IPTG and arabinose simultaneously and incubated for 24 hours, while FIG. 14B illustrates decasaccharide (DP4) as major peak obtained when such a culture is induced first with IPTG and incubated for 24 hours, followed by induction of eliminase with arabinose and incubated for further 24 hours. That is, a significant difference may he observed when heparosan and eliminase production is induced together or sequentially with ˜24 hours differences in the induction. When they are induced together and cultured for 24 hours, oligosaccharides, ranging from tetrasaccharide to dodecasaccliarides, may be obtained with tetrasaccharide being the major product, an example of which is shown in FIG. 14A. When an eliminase gene is induced 24 hours after induction of heparosan biosynthesis and continued for additional 24 hours of culture, decasaccharide as the major peak may be observed followed by octasaccharide and then hexasaccharide along with small amounts of higher oiigosaccharides, an example of which is shown in FIG. 14B. In this manner, oligosaccharide chain length and their relative yields may be successfully controlled for the first time through differential induction of the heparosan production and eliminase expression (concurrent, or stepwise) and controlling culture conditions (temperature and duration of culture).

Conclusions. Heparosan is an essential starting material for the production of heparin-like structures to study structure-function relationships and for preparation of recombinant LMWH anticoagulants. Using one or more of the methods described herein to clone heparosan biosynthetic gene clusters in various orders from E. coli K5 into different non-pathogenic E. coli strains; e.g., BL21(DE3), HT115(DE3), and Shuffle T7 Express B, the five distinct strains discussed above may be metabolically engineered to prepare recombinant heparosan polysaccharides with yields up to ˜480 mg/L of recombinant heparosan. Also, by changing media composition and duration of culture, the molecular weights of heparosan produced may be controlled, with the weights ranging from about 5 kDa to >150 kDa. Also using the methods described herein, the heparosan biosynthetic genes may be co-transformed together with E. coli K5 elmA gene, and can be differentially induced using IPTG (for heparosan) and arabinose (for eliminase), and thereby, achieved a controlled production of heparosan oligosaccharides of four specific sizes (DP4, DP6, DP8, and DP10) as predominant oligosaccharides for the first time. The ability to produce different oligosaccharide sizes is advantageous as the biological activity of heparin-like oligosaccharides is size-dependent; thus, various oligosaccharide sizes are required for studying the binding of oligosaccharides to proteins. The results, as described herein, enable the production of biologically active heparin-like structures using recombinantly generated heparosan structures as synthons.

Example Steps for Practicing the Example Methods According to the Embodiments of this Disclosure

Vectors and Bacterial Strains:

E. coli K5 and DH5α were obtained from American Type Culture Collection (ATCC), and E. coli HT115 was a kind gift of Dr. Sanchez Alvarado (Stowers Institute for Medical Research). E. coli BL21(DE3) and chemically competent E. coli Shuffle T7 Express B (a mutated BL21 DE3 strain) were obtained from New England Biolabs. Chemically competent E. coli cells were prepared by the rubidium chloride method and were used for sub-cloning and plasmid constructions. Plasmids, pETDuet-1, pRSFDuet-1, and pRADmyc/HisA with PBAD promoter were obtained from Invitrogen. Restriction enzymes and DNA ligases were from Promega and New England Biolabs. Culture media, chromatographic materials, solvents, common chemicals and biochemical were purchased from Sigma Aldrich and Fisher Scientific.

Plasmid Construction, Sub-Cloning, and Bacterial Strains:

E. coli K5 genomic DNA was prepared using the Purelink genomic DNA mini kit (Invitrogen). Genes of KfiA, KfiB, KfiC, KfiD, KfiAB, KfiCD, and elmA were cloned from the genomic DNA of E. coli K5 using PCR with Platinum Pfx DNA Polymerase (Invitrogen) or Phusion High-Fidelity PCR Master Mix (ThermoFisher Scientific), and primers are shown in Table 1. The PCR products were incubated with dATPs and Tag Polymerase (Promega) to add A-overhangs and were then sub-cloned into pCR2.1 vectors using TOPO-TA cloning kit (Invitrogen). The plasmids were, then, digested using the restriction enzymes shown in Table 1. The expression plasmids were also digested with the respective enzymes, as shown in Table 1. The elmA fragment was inserted into pBAD-myc-HisA to prepare a pBAD-elmA construct. Fragments of KfiA and KfiAB were cloned into the multi-cloning site 1 (MCS1) of pETDuet-1 to create constructs pET-KfiA and pET-KfiAB, respectively.

TABLE 1
Primers and Expression Plasmids

| | | | Restriction |
|---|---|---|---|
| Gene | Primers | Plasmid | Enzymes |
| KfiA | KfiA-F: | pET-Duet1 | NdeI, XhoI |
| | AGAAGGAGATATACATATGATGATTGTTGCAAATATGTCA | | |
| | KfiA-R: | | |
| | GTTTCTTTACCAGACTCGAGTTACCCTTCCACATTATACA | | |
| KfiB | KfiB-F: | pET-Duet1 | NdeI, XhoI |
| | AGAAGGAGATATACATATGATGATGAATAAATTAGTGCTA | | |
| | KfiB-R: | | |
| | GTTTCTTTACCAGACTCGAGTTAACAGCCCTTGATTTTAG | | |
| KfiC | KfiC-F: | pRSF-Duet1 | NcoI, SacI |
| | AAGAAGGAGATATACCATGGGCATGAACGCAGAATATATA | | |
| | KfiB-F: | | |
| | AGAAGGAGATATACATATGATGATGAATAAATTAGTGCTA | | |
| KfiD | KfiD-F: | pRSF-Duet1 | NcoI, SacI |
| | AATAAGGAGATATACCATGGGCATGTTCGGAACACTAAAA | | |
| | KfiD-R: | | |
| | TGCAGGCGCGCCGAGCTCTTAGTCACATTTAAACAAATCG | | |
| KfiAB | KfiA-F: | pET-Duet1 | NdeI, XhoI |
| | AGAAGGAGATATACATATGATGATTGTTGCAAATATGTCA | | |
| | KfiB-R: | | |
| | GTTTCTTTACCAGACTCGAGTTAACAGCCCTTGATTTTAG | | |
| KfiCD | KfiC-F: | pRSF-Duet1 | NcoI, SacI |
| | AAGAAGGAGATATACCATGGGCATGAACGCAGAATATATA | | |
| | KfiD-R: | | |
| | TGCAGGCGCGCCGAGCTCTTAGTCACATTTAAACAAATCG | | |
| ElmA | ElmA-F: AAGTCCATGGCGGTCTCAACCGAA | pBAD- | EcoRI, NcoI |
| | ElmA-R: AAGGAATTCAATTCCCTGTTAATTGCAAAACT | myc/hisA | |

The KfiD and KfiCD fragments were placed into the MCS2 of pRSFDuet-1 to create pRSF-KfiC and pRSF-KfiCD, respectively (FIGS. 9A and 9B). pET-KfiA was restriction digested, and its MCS2 may be used to insert KfiC to prepare pET-KfiC-KfiA (FIG. 3A). Similarly, pRSF-KfiD was restriction digested, and its MCS1 was used to insert KfiB to prepare pET-KfiD-KfiB (FIG. 3B). pRSF-KfiCD-KfiAB was generated by inserting the KfiAB fragment from pET-KfiAB into the MC2 site of pRSF-KfiCD. Thus, six final plasmid constructs: pET-KfiC-KfiA, pRSF-KfiD-KfiB, pET-KfiAB, pRSF-KfiCD, pRSF-KfiCD-KfiAB and pBADmyc/hisA-elmA (FIGS. 7A-9B and 2) were engineered in this manner. All PCR-cloned genes were verified by sequencing (GeneWiz).

The pET-KfiAB (FIG. 4A) and pRSF-KfiCD (FIG. 4B) were co-transformed into E. coli strains BL21 (DE3), HT115 (DE3) and Shuffle-T7B(DE3) to create recombinant strains BL21-CDAB, HT115-CDAB, Shuffle-CDAB, respectively, as shown in FIG. 2. Plasmid constructs pET-KfiA-KfiC (FIG. 3A) and pRSF-KfiB-KfiD (FIG. 3B) were co-transformed into E. coli BL21 (DE3) and HT115 (DE3) to create strains BL21-CADB and HT115-CADB, respectively, as shown in FIG. 2, pRSF-KfiCD-KfiAB (FIG. 12A) and pBAD-myc/hisA-elmA (FIG. 12B) plasmids were co-transformed into BL21(DE3) strain to create BL21-CDAB-elmA, as shown in FIG. 1.

Media Conditions:

Modified M9 minimal medium (1× M9 sails, 2 mM MgCl₂, 0.1 mM CaCl₂, 0.4% Glycerol, 1 mM Taurine) and glycerol-based Nutrient-Rich Media (20.0 g casamino Acids, 4.8 g NaH₂PO₄, 5.4 g K₂HPO₄, 4.2 g KH₂PO₃, 0.5 g MgSO₄, 0.018 g FeSO₄, 1.0 g NaCl, 0.25 g Na₂SO₄, 15 mL glycerol, 3.0 mL vitamin buffer (see below), 10 mL of nutrient buffer (see below) per one litter) were used as culture medium in shake-flask and 6 L bioreactor cultures. One liter of 5× M9 salts contain 64 g NaHPO₄, 7H₂O, 15 g KH₂PO₄, 2.5 g NaCl and 5 g NH₄Cl. One liter of vitamin buffer contains 0.21 g riboflavin, 0.167 g thiamine, 2.7 g pantothenic acid, 3.0 g niacin, 0.7 g pyridoxine, 0.03 g biotin, 0.02 g folic acid. One liter of the nutrient buffer contains 50 g of (NH₄)₂SO₄ and 100 mL of trace element mixture. One liter of trace element mixture contains 0.05 g H₃PO₄, 0.0025 CuSO₄, 0.2 g ZnCl₂, 1.2 g CoCl_{2 6}H₂O, 0.25 g AlCl₃, 0.2 g Na₂MoO₄. M9 salts, trace element solution, vitamin buffer, and nutrient buffer were filtered with 0.2 μm sterile filtration unit and added to respective autoclaved media. Appropriate antibiotics (ampicillin (100 mg/L, kanamycin 50 mg/L) were used.

Shake Flask and Bioreactor Cultures:

After bacteria were cultured in LB medium for overnight, 1% of the culture was subcultured in nutrient-rich medium or M9 medium in 1 L shake flasks at 225 rpm and 37° C. Expression of KfiA, KfiB, KfiC, and KfiD was induced with isopropyl β-D-1-thiogalactopyranoside (IPTG, 0.1 mM) at 0.4-0.6 of OD₆₀₀. The E. coli were harvested after 24 hours of culture for polysaccharide purification. The 6-L fed-batch culture was performed in a 10 L Fermentas Bioreactor. 60 ml of overnight LB culture was used as inoculant and cultured at 37° C. and 350 rpm. The pH of the medium was adjusted at 6.8 using NFL₄OH. IPTG was added to a final concentration of 0.1 mM after culture has reached OD600 of 0.5-1.0 (depending on different sets of experiments). This was followed by addition of 60 mL glycerol, 1 hour after IPTG induction, and then glycerol was pumped into the bioreactor at a rate of 1.8 mL/hour. The culture was then harvested.

Isolation of Polysaccharides:

After harvesting the culture, cells were autoclaved at 121° C. for 30 minutes, and then the cultures were allowed to cool. CaCl₂ and protease were then added to make a final concentration of 1 mM and 100 mg/L, respectively. Following the protease addition, the mixture was incubated at 37° C. with shaking at 75 rpm for 15 hours. Next, the mixture was centrifuged at 15000 g at room temperature for 30 minutes to remove cell debris, and the supernatant was filtered through 0.22 μm PES filter (ThermoFisher).

Polysaccharide isolation columns were prepared using DEAE-sepharose beads (50 mL/L culture media) and washed with 3 column volumes of water followed by equilibration with 3 column volumes of wash buffer (20 mM NaOAc, 0.1 M NaCl, pH 6.8). The filtrate of the supernatant was diluted with an equal volume of the wash buffer and loaded three times on the DEAE-sepharose column. The columns were, then, washed with 30 column volumes of the wash buffer, followed by elution with 6 column volumes of elution buffer (20 mM NaOAc, 1 M NaCl, pH 6.8). The elution was then concentrated using 3000 MWCO dialysis unit (Sartorius). Absolute ethanol was added to the concentrate to make 80% final concentration of ethanol, and it was kept at 4° C. overnight. The solution was centrifuged at 4000 rpm at 4° C. for 60 minutes, and the pellet was dried completely. To remove DNA, the dry pellet was then dissolved in DNase buffer (100 mM Tris, 1 mM MgCl₂, 0.5 mM CaCl₂, pH 7.5). and treated with freshly prepared DNase solution (1 mg DNase/L final concentration) and the mixture was incubated at 37° C. with shaking at 75 rpm for 15 hours, This step was followed by the addition of protease (5 mg/L) and further incubation at 56° C. with shaking at 75 rpm for 2 hours. The solution was then dialyzed using 3000 MWCO dialysis unit and then frozen at −80° C. The frozen samples were lyophilized, and the dry weights were recorded.

Extraction and Purification of Oligosaccharides:

The E. coli BL21-CDAB-elmA strain was cultured at 37° C. and 200 rpm in nutrient-rich medium with both ampicillin and kanamycin. The plasmid construct pRSF-KfiCD-KfiAB was induced with IPTG at the final concentration of 100 μM, and pBAD-myc/hisA-elmA was induced with arabinose at the final concentration of 0.1% w/w.

The plasmid expression was induced in two different conditions: A and B. Condition A was induced with IPTG at 0.4-0.6 of OD₆₀₀ and incubated for 24 hours, followed by arabinose induction and cultured for another 24 hours. Condition B was simultaneously induced with IPTG and arabinose at 0.4-0.6 of OD₆₀₀ and incubated for 24 hours or 48 hours. The cultures were repeated with different temperature conditions and time lengths of incubation after induction with IPTG.

After the cultures were harvested, they were centrifuged at 5000 rpm and 4° C. for 30 rain. The bacterial pellet was re-suspended with 150 ml of H2O. The solution was centrifuged at 5000 rpm at room temperature for 30 min. The pellet was resuspended in 10 ml of H2O and sonicated for 90 seconds with an interval of one second on and one second off. The lysed solution was centrifuged at 4000 rpm and 4° C. for 30 min, and the supernatant was used for further purification. Five column volumes of methanol followed sequentially by 1M HCl, and H2O were used to wash 4 g of Amberlite H+resin in 15 ml tube before loading to a 1.0×10 cm glass column (GE Healthcare). The resin was further rinsed with ten column volumes of H2O. The lysed solution was loaded once, and the flow-through was collected and centrifuged at 4000 rpm for 10 min at room temperature. The supernatant was transferred to a new tube, and the pH was adjusted to 7.0 with 1M NaOH solution.

Structural Analysis of Heparosan:

For quantification of heparosan in terms of glucuronic acid content in the samples, the colorimetric assay, carbazole assay, was carried out using a UV/Vis spectrophotometer. Lyophilized heparosan was dissolved in water and digested with heparinase I (in presence of heparinase buffer) overnight or for a very short period (less than 5 min), before heat inactivation to stop the reaction, to obtain disaccharides or oligosaccharides respectively. These were analyzed by HPLC (Hitachi) on a Carbopac analytical column. A gradient of 0 M NaCl, 20 mM NaOAc, pH 3.5 to 2 M NaCl, 20 mM NaOAc, pH 3.5 over 75 minutes was used to resolve and isolate the structures. The isolated disaccharides and oligosaccharides were analyzed using a capillary C-18 column in the Liquid Chromatography coupled to Mass Spectrometry Unit (Bruker ESI-qTOF). Moreover, for ¹H NMR spectra, 10 mg of the lyophilized samples were dissolved in 1 mL D₂O and deuterium-hydrogen exchange was carried out three times before conducting ¹H NMR studies by a Mercury 500 MHz NMR spectrometer.

The molecular weight distributions of the samples were analyzed by HPLC Size Exclusion Chromatography using two G300005WXL Columns (Tosoh, 7.8 mm×30 cm) connected back-to-back in tandem and by eluting over 60 min with phosphate buffer (100 mM KH2PO4, 100 mM NaCl, pH 6). The average molecular weight was calculated by running polystyrene sulfonate standards and calculating their migration times. For time point experiments, 200 mL culture was taken from a 2 L shake flask at 12, 24, 36 and 48 hours. Each sample was immediately autoclaved and heparosan was extracted as discussed above.

Cloning of Eliminase A from E. coli K5:

Genomic DNA was prepared from E. coli K5 (ATCC) using PureLink genomic DNA kit (Invitrogen). Forward primers used were elmA-S1 (SEQ ID NO: 4), elmA-S2 (SEQ ID NO: 5), and elmA-S3 (SEQ ID NO: 6) with one common reverse primer elmA-AS (SEQ ID NO: 7) to clone different open reading frames (ORFS) of the gene Eliminase (elmA, GenBank: X96495.1). These were subsequently named as elmA-1 (SEQ ID NO: 1), elmA-2 (SEQ ID NO: 2), and elmA-3 (SEQ ID NO: 3). PCR was carried out using Phusion High-Fidelity PCR Master Mix (ThermoFisher Scientific) and was verified using 1% agarose gel electrophoresis.

E.Z.N.A. Cycle Pure kit (Omega) was used for PCR clean-up. The plasmid pBAD-myc/HisA with P_{BAD} promoter was obtained from Invitrogen. The purified PCR products and plasmid were digested by EcoR1 and Nco1 (New England Biolabs) and gel extraction was done using Monarch Gel Extraction Kit (New England Biolabs) to get sticky-ended elmA-1, elmA-2, and elmA-3 and linearized pBAD-myc-HisA, respectively. Ligation was done using Anza Ligase Mix (ThermoFisher Scientific) and transformed into chemically-competent E. coli D115α. Colony PCR was carried out with Green Taq polymerase (Promega) to verily positive colonies, followed by plasmid preparation using Plasmid DNA mini-kit (Omega). The plasmids were sequenced using pBAD forward primer at GeneWiz. Thus, three constructs were prepared pBAD-myc/HisA-elmA-1 (FIG. 15), pBAD-myc/HisA-elmA-2 (FIG. 16) and pBAD-myc/HisA-elmA-3 (FIG. 17). The constructs were transformed into competent E. coli BL21 DE3 or Top10 strains to make three strains elmA-s1, eltmA-s2, and elmA-s3, respectively. All transformations were done in LB agar plates with 0.1 mg/mL ampicillin and cultures were grown in LB medium with 0.1 mg/mL ampicillin. Glycerol stocks were prepared with 25% glycerol.

Expression and Purification of Enzymes:

Fresh colonies or glycerol stocks were used to inoculate overnight cultures of BL21 DE3 or Top10 strains, which were used to start 500 ml. LB medium cultures (with 0.1 mg/mL ampicillin). They were grown at 37° C. until OD₆₀₀ reached 0.4-0.5 followed by induction done using 0.1% arabinose. The induced culture was left overnight at 25° C. The cultures were then centrifuged at 4000 rpm at 4° G for 30 min and the supernatant was decanted (no enzyme was found in the medium). The cell pellet was re-dissolved in 50 ml. lysis buffer (100 mM Tris. pH 7.5. 25% w/v sucrose, 1 mM PMSF, 0.1 mg/mL DNase, 1 mg mL lysozyme) and left on ice for 30 min, TritonX-100 (1%) was added and the lysate was then sonicated and subsequently centrifuged at 15000 rpm for 30 min at 4° C. The clear lysate was filtered through 0.22 μM filters (Whatman) and the filtrate was used for enzyme purification.

The cell lysate was diluted with ice-cold buffer A (100 mM Tris pH 7.5, 500 mM NaCl, 20 mM imidazole, 5% glycerol, 2 mM 2-mercaptoethanol) and run through a gravity column with 5 mL nickel resin (GE Healthcare) three times in a cold room. The column was washed with 30 column volumes of buffer A and the enzyme was eluted with 7 column volumes of buffer B (100 mM Tris pH 7.5, 500 mM NaCl, 500 mM imidazole, 5% glycerol, 2 mM 2-mercaptoethanol). The eluate was concentrated with a 3000 MWCO filter and buffer exchanged with 10 mM PIPES buffer with 5% glycerol. Subsequently BCA assay s were performed to find enzyme concentrations and each eliminase A enzyme could be stored at 4° C. for up two weeks before use or stored at −20° C. for extended period of time.

### SUPPLEMENTAL EXAMPLES

### Results of Trials with Additional Recombinant E. coli Strains and Assays

The methods described herein to may be used to the clone heparosan biosynthetic genes in additional orders into different non-pathogenic E. coli strains; e.g., BL21(DE3), HT115(DE3), and Shuffle T7 Express B, to produce additional distinct strains that are metabolically engineered to prepare recombinant heparosan polysaccharides. The following example illustrates outcomes that may be obtained with the gene order KfiABCD to obtain five additional distinct strains, which yielded recombinant heparosan with high yield and controllable chain length ranging from 5-6 kDa to >70 kDa, which demonstrated controlled and easily scalable preparation of large amounts of size-defined oligosaccharides.

In another example constructs pET-KfiAB and pRSF-KfiCD were co-transformed into each of E. coli strains including BL21(DE3), HT115(DE3) and Shuffle T7 B to create strains BL21 AB-CD, HT115 AB-CD and Shuffle AB-CD. Likewise, pET-KfiA-KfiC and pRSF-KfiB-KfiD were co-transformed into E. coli BL21(DE3) and HT115(DE3) strains to create BL21 AC-BD and HT115 AC-BD. These strains were cultured using conditions similar to those described above with respect to strains discussed above.

Shake flask cultures with M9 modified medium gave the highest amount of recombinant heparosan for the construct BL21-ABCD (FIG. 18A). Time dependence of expression showed that highest levels of heparosan were formed at 36 h for BL21-ABCD (FIG. 18B). The 6 L batch bioreactor cultures of BL21-ABCD gave an average of 100 mg/L heparosan (FIG. 18C). While M9 medium provided easy-to-clean samples, the quantity was much lower. When Terrific Broth medium was used, the samples retained substantial impurities, most probably because it contains a high amount of tryptone (partially hydrolyzed casein protein). Hence, the glycerol-based nutrient-rich medium with casamino acids (completely hydrolyzed casein protein) was next used as the protein source. This increased yield while reducing the impurities. Since highest levels of heparosan were expressed for BL21-ABCD at the 24 h time point in the shake flask method (FIG. 18A), the same using modified enriched medium was used to grow 6 L bioreactor batch culture with glycerol fed at 2 mL/hour (50%). This culture with 1 mM IPTG at OD₆₀₀ of 18.0 and yielded a heparosan amount of 500 mg/L (FIG. 8B).

Quantification of heparosan obtained with strains BL21 AB-CD, HT115 AB-CD, Shuffle AB-CD, BL21 AC-BD, and HT115 AC-BD was carried out using a carbazole assay, substantially as described above with the previously-discussed strains, using a UV/Vis spectrophotometer. Recombinant heparosan from all strains gave very similar disaccharide profiles resembling that from E. coli KS. Treatment of E. coli BL21 AB-CD derived heparosan treated with heparinase I and run through a Carbopac column in HPLC showed several well-isolated peaks. Collection of these peaks followed by MS analysis showed molecular weights (FIG. 19B is a z1 profile of DP2; FIGS. 19C-19F are z2 profiles of DP4, DP6, DP8, and DP10, respectively) as the standard oligosaccharide profile. The structure of heparosan from E. coli K5 and recombinant strains E. coli BL21 DE3 (BL21-AB-CD and BL21-AC-BD), E. coli HT115 DE3 (HT115-AB-CD and HT115-AC-BD) and E. coli Shuffle T7 Express B (ShuffleT7-AB-CD) was studied using ¹H NMR spectroscopy. The spectra were found to be nearly identical (FIGS. 20A-20C). However, recombinant heparosan derived from either E. coli BL21-AB-CD or Shuffle AB-CD was found to be cleanest, The chemical shift data of heparosan from BL21-AB-CD and that from K5 is shown in Table 2.

TABLE 2
Chemical shift assignments of heparosan from E. coli K5
and recombinant E. coli BL21 DE3 (BL21 AB-CD) in ppm (±0.005)

| | | Chemical Shift | Chemical Shift |
|---|---|---|---|
| ¹H NMR | Proton | for K5 | for BL21AB-CD |
| 1 | Methyl H of GlcNAc | 1.898 | 1.885 |
| 2 | H-2 atom of GlcA | 3.219 | 3.206 |
| 3 | H-4 atom of GlcNAc and | 3.546 | 3.572 |
| | H-3, H-4, H-5 atom of GlcA | | |
| 4 | H-2, H-3, H5, H-6 atom | 3.710 | 3.698 |
| | of GlcNAc | | |
| 5 | H-1 atom of GlcA | 4.347 | 4.338 |
| 6 | H-1 atom of GlcNAc | 5.249 | 5.235 |

The SEC profiles of K5 and recombinant heparosans were analyzed using the elution pattern of polysulphonated standards (FIGS. 21 (standards), 22, and 23). The BL21-ABCD, HT115-ABCD, BL21-ACBD, HT115-ACBD and ShuffleT7-ABCD-derived heparosans from shake flask cultures of 24 h gave SEC profiles corresponding to an average molecular weight of 5000 Da (line 18 of FIG. 22). The BL21-ABCD culture prepared in bioreactor for 48 h and enriched with glycerol (50%, 2 ml/hr.) yielded a mixture of two products including 5000 Da and 30 kDa (line 16 of FIG. 22). Recombinant heparosan prepared in BL21-ABCD cultures grown in M9 modified culture medium also yielded two products, but with average molecular weights corresponding to longer chains, i.e., 30-40 kDa and >70 kDa (more than exclusion limit of column). The expression of these chains varied significantly with the time of culture with the larger product increasing with time (FIG. 23: 6 hr. culture time, line 20; 12 hr. culture time, line 22; 24 hr. culture time, line 24; 36 hr. culture time, line 26; and 48 hr. culture time, line 28). In comparison, non-recombinant heparosan from E. coli K5, gave an average molecular weight of about 30000 Da.

Enzymatic reactions for preparation of heparosan oligosaccharides: FIGS. 24-34B illustrate example outcomes of enzymatic reactions for preparation of heparosan oligosaccharides, in which the oligosaccharides DP6 and DP8 were isolated using HPLC and treated with two key HS biosynthetic enzymes including C5-epimerase (EPI) to yield appropriate DP6 and DP8 oligosaccharides related to HS showing the success in synthesis of these sulfated, epimerized oligosaccharides. FIG. 24 illustrates a pathway for the production of heparin-like anticoagulant therapies and chemo-enzymatic synthesis of heparin-like polysaccharides.

In one example, heparin-like structures of specific size and sulfation patterns were produced as described with respect to FIGS. 25A-26F. Recombinant heparosan was treated chemically to make it undergo N-deacetylation/N-sulfation and 6-O-sulfation. Recombinant biosynthetic enzymes: C5-epimerase, 2-O-sulfotransferases, 3-O-sulfotransferases were used to create structures C, D, F, G, H from FIG. 24. The final constructs were digested with heparin I, II, III before analyzing.

FIGS. 25A-26F illustrate results of LC/MS disaccharide analysis of the heparin-like structures of specific size and sulfation patterns that resulted of chemical/enzymatic treatment of recombinant heparosan. FIGS. 25A-25D are HPLC chromatograms of disaccharides of: heparosan (FIG. 25A), heparosan after chemical N-deacetylation/N-sulfation (FIG. 25B), enzymatic 2-O-sulfation of NS-C5Epi-heparosan (FIG. 25C), and chemical 6-O-sulfation of NS-C5Epi-heparosan (FIG. 25D). FIGS. 26A-26F are mass-spectrometric characterizations of: z1 of heparosan (FIG. 26A), disaccharide (FIG. 26B), z1 of NS-heparosan (FIG. 26C), z2 of DP8 NS-heparosan after epimerization and showing 1 deuterium addition (FIG. 26D), Z1 of 3-O-, 6-O-sulfated NS-epimerized heparosan DP2 (FIG. 26E), and z2 peaks of trisulfated and tetrasulfated oligos (FIG. 26F).

1 ug of enzyme (either elmA-1, elmA-2, or elmA-3) was used to treat 100 μg of heparosan in presence of 50 mM Tris. pH 7.5-8.0. The eliminase reaction was allowed to continue at 37° C. with periodic monitoring. After 72 h of incubation, the analysis was performed using HPLC, as described above. The products revealed the presence of heparosan oligosaccharides of DP4 to DP10 (FIG. 27). As the digestion progressed, the proportions of DP6 and DP8 were found to be the highest. The MS analysis of these oligosaccharides indicated the structure to be oligomeric entities derived from heparosan's scaffold and containing a double bond at the non-reducing terminus (FIG. 28). These oligosaccharides could he readily separated into individual homogeneous products of DP4, DP6, DP8, DP10. etc. Importantly, disaccharides were not observed.

FIGS. 29-30EE illustrate results of size-controlled oligosaccharide production in which the elmA from E. coli K5 was cloned to the bacterial strains Top10 and BL21 DE3, as described above, with hexasaccharide and octasaccharide being the major products. FIG. 29 is a time-dependent digestion profile of the oligosaccharides prepared from recombinant eliminase enzyme from the metabolically engineered Top10 E. coli on days 10-14 (shown in FIG. 29: day 10 line 30; day 11, line 32; day 12, line 34; day 13, line 36, and day 14, line 38). FIGS. 30A-30E are mass spectrometer characterizations of the oligosaccharide digestion from days 10-14 with recombinant Eliminase enzyme prepared from metabolically engineered Top 10 E. coli.

FIGS. 31A and 31B are mass spectrometer characterizations of the oligosaccharide profiles after digestion with recombinant eliminase enzyme prepared from the metabolically engineered. BL21 DE3 (FIG. 31A) and Top10 (FIG. 31B) strains. FIGS. 32A-32D are characterizations of oligosaccharides: tetra- hexa-, octa-, and deca-saccharides, respectively, after separation through prep-HPLC and confirmation through mass spectrometry.

FIGS. 33A and 33B are mass spectrometer characterizations of hexasaccharide after chemical N-deacetylation/N-sulfation (FIG. 31A) and after epimerase treatment (FIG. 33B). FIGS. 34A and 34B are mass spectrometer characterizations of octasaccharide after chemical N-deacetyation/N-sulfation (FIG. 34A) and after epimerase treatment (FIG. 34B).

The description of the invention and its applications as set forth herein is illustrative and is not intended to limit scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

What is claimed is:

1. A method for producing heparosan oligosaccharides, the method comprising:
culturing a recombinant host cell of a non-pathogenic bacterial strain, the recombinant host cell having been engineered to comprise the biosynthetic gene cluster KfiA, KfiB, KfiC, KfiD and the gene elmA, in culture conditions enabling direct expression of heparosan oligosaccharides by the recombinant host cell; and
obtaining a plurality of heparosan oligosaccharides expressed by the recombinant host cell during the culturing,
wherein a majority of the plurality of heparosan oligosaccharides range in size from approximately tetrasaccharide to approximately dodecasaccharide.

2. The method of claim 1, further comprising inducing the recombinant host cell to express heparosan via the biosynthetic gene cluster approximately 24 hours before inducing the host cell to express eliminase via elmA.

3. The method of claim 2, wherein the dodecasaccharide size is the most prevalent size among the plurality of heparosan oligosaccharides.

4. The method of claim 1, further comprising inducing the recombinant host cell to express heparosan via the biosynthetic gene cluster approximately concurrently with inducing the host cell to express eliminase via elmA.

5. The method of claim 4, wherein the tetrasaccharide size is the most prevalent size among the plurality of heparosan oligosaccharides.

6. The method of claim 1, wherein the gene elmA comprises one of the isoforms elmA-2, or elmA-3.

7. The method of claim 1, wherein the non-pathogenic bacterial strain comprises one of BL21 -CDAB, HT115-CDAB and Shuffle-CDAB, and wherein the genes of the gene cluster KfiA, KfiB, KfiC, KfiD have been cloned into the recombinant host cell in the order KfiC, KfiD, KfiA, KfiB using one or more vectors.

8. The method of claim 1, wherein the non-pathogenic bacterial strain comprises one of BL21-CDAB, HT115-CDAB and Shuttle-CDAB, and wherein the genes of the gene cluster KfiA, KfiB, KfiC, KfiD have been cloned into the recombinant host cell in the order KfiA, KfiB, KfiC, KfiD using one or more vectors.

9. The method of claim 1, wherein the non-pathogenic bacterial strain comprises one of BL21-CDAB, HT115-CDAB and Shuffle-CDAB, and wherein the genes of the gene cluster KfiA, KfiB, KfiC, KfiD have been cloned into the recombinant host cell individually or with gene pairs KfiAB and KfiCD using one or more vectors, in any order or as a single cluster KfiABCD.

10. The method of claim 1. wherein culturing the recombinant host cell comprises culturing the recombinant host cell at one or more temperatures within a range of approximately 14 degrees Celsius to approximately 40 degrees Celsius.

11. The method of claim 11, wherein culturing the recombinant host cell comprises culturing the recombinant host cell at a plurality of the temperatures within the range of approximately 14 degrees Celsius to approximately 40 degrees Celsius.

12. A recombinant host cell of a non-pathogenic bacterial strain, the recombinant host cell having been engineered to comprise the biosynthetic gene cluster KfiA, KfiB, KfiC, KfiD and the gene elmA,
wherein the recombinant host cell is engineered to directly express a plurality of heparosan oligosaccharides when cultured under suitable conditions, and
wherein a majority of the plurality of heparosan oligosaccharides range in size from approximately tetrasaccharide to approximately dodecasaccharide.

13. The recombinant host cell of claim 12, wherein the dodecasaccharide size is the most prevalent size among the plurality of heparosan oligosaccharides.

14. The recombinant host cell of claim 12, wherein the tetrasaccharide size is the most prevalent size among the plurality of heparosan oligosaccharides.

15. The recombinant host cell of claim 12, wherein the gene elmA comprises one of the isoforms elmA-1, elmA-2, or elmA-3.

16. The recombinant host cell of claim 12, wherein the non-pathogenic bacterial strain comprises one of BL21-CDAB, HT115-CDAB and Shuffle-CDAB, and wherein the genes of the gene cluster KfiA, KfiB, KfiC, KfiD have been cloned into the recombinant host cell in the order KfiC, KfiD, KfiA, KfiB.

17. The recombinant host cell of claim 12, wherein the non-pathogenic bacterial strain comprises one of BL21-CDAB, HT115-CDA13 and Shuffle-CDAB, and wherein the genes of the gene cluster KfiA, KfiB, KfiC, KfiD have been cloned into the recombinant host cell in the order KfiA, KfiB, KfiC, KfiD using one or more vectors.

18. The recombinant host cell of claim 12, wherein the non-pathogenic bacterial strain comprises one of BL21-CDAB, HT115-CDAB and Shuffle-CRAB, and wherein the genes of the gene cluster KfiA, KfiB, KfiC, KfiD have been cloned into the recombinant host cell individually or with gene pairs KfiAB and KfiCD using one or more vectors, in any order or as a single cluster KfiABCD.

19. The recombinant host cell of claim 12, wherein the suitable conditions comprise one or more temperatures within a range of approximately 14 degrees Celsius to approximately 40 degrees Celsius.

20. The recombinant host cell of claim of claim 19, wherein the suitable conditions comprise a plurality of the temperatures within the range of approximately 14 degrees Celsius to approximately 40 degrees Celsius.

21. A method for producing heparosan polysaccharides, the method comprising:
culturing a recombinant host cell of a non-pathogenic E. coli strain, the recombinant host cell having been engineered to comprise the biosynthetic gene cluster KfiA, KfiB, KfiC, KfiD, in culture conditions enabling direct expression of heparosan polysaccharides by the recombinant host cell; and
obtaining a plurality of heparosan polysaccharides expressed by the recombinant host cell during the culturing,
wherein a majority of the plurality of heparosan polysaccharides range in mass from approximately 900 Da to approximately 5 KDa,

22. The method of claim 21, wherein a majority of the plurality of heparosan polysaccharides range in mass from approximately 5 KDa to approximately 30 KDa, 23, The method of claim 22, wherein culturing the recombinant host cell comprises culturing the recombinant host cell in nutrient-rich medium comprising casamino acids and glycerol in a shake-flask.

24. The method of claim 23, wherein the approximately 5 KDa or 900 Da to 5 KDa mass is the most prevalent mass among the plurality of heparosan polysaccharides.

25. The method of claim 21, wherein culturing the recombinant host cell comprises culturing the recombinant host cell in nutrient-rich medium comprising casamino acids and glycerol under fed-batch conditions inn a bioreactor.

26. The method of claim 25, wherein a majority of the plurality of heparosan polysaccharides comprise a mass of approximately 5 KDa or a mass of approximately 30 KDa.

27. The method of claim 25, wherein the nutrient-rich medium comprises 50% glycerol, the method further comprising adding glycerol to the culture at 1.8 mL per hour.

28. The method of claim 21, wherein the non-pathogenic bacterial strain comprises one of BL21-CDAB, HT115-CDAB and Shuffle-CDAB, and wherein the genes of the gene cluster KfiA, KfiB, KfiC, KfiD have been cloned into the recombinant host cell in the order KfiC, KfiD, KfiA, KfiB using one or more vectors.

29. The method of claim 21, wherein the non-pathogenic bacterial strain comprises one of BL21-CDAB, HT115-CDAB and Shuffle-CDAB, and wherein the genes of the gene cluster KfiA, KfiB, KfiC, KfiD KfiB, have been cloned into the recombinant host cell in the order KfiA, KfiB, KfiC, KfiD using one or more vectors.

30. The method of claim 21, wherein the non-pathogenic bacterial strain comprises one of BL21-CDAB, H1115-CDAB and Shuffle-CDAB, and wherein the genes of the gene cluster KfiA, KfiB, KfiC, KfiD have been cloned into the recombinant host cell individually or with gene pairs KfiAB and KfiCD using one or more vectors, in any order or as a single cluster KfiABCD.

31. The method of claim 21, wherein the culturing comprises culturing the recombinant host cell at one or more temperatures within a range of approximately 14 degrees Celsius to approximately 40 degrees Celsius.

32. The method of claim 31, wherein the culturing comprises culturing the recombinant host cell at a plurality of the temperatures within the range of approximately 14 degrees Celsius to approximately 40 degrees Celsius.

33. A recombinant host cell of a non-pathogenic bacterial strain, the recombinant host cell having
been engineered to comprise the biosynthetic gene cluster KfiA, KfiB, KfiC, KfiD
wherein the recombinant host cell is engineered to directly express a plurality of heparosan polysaccharides when cultured under suitable conditions, and
wherein a majority of the plurality of heparosan polysaccharides range in size from approximately 900 KDa to approximately 30 KDa.

34. The recombinant host cell of claim 33, wherein a majority of the plurality of heparosan polysaccharides range in mass from approximately 5 KDa to approximately 30 KDa.

35. The recombinant host cell of claim 33, wherein the suitable conditions comprise culturing the recombinant host cell in nutrient-rich medium comprising casamino acids and glycerol in a shake-flask.

36. The recombinant host cell of claim 35, wherein the approximately 5 KDa mass is the most prevalent mass among the plurality of heparosan polysaccharides.

37. The recombinant host cell of claim 33, wherein the suitable conditions comprise culturing the recombinant host cell in nutrient-rich medium comprising casamino acids and glycerol under fed-batch conditions in a bioreactor.

38. The recombinant host cell of claim 37, wherein a majority of the plurality of heparosan polysaccharides comprise a mass of approximately 5 KDa or a mass of approximately 30 KDa.

39. The recombinant host cell of claim 37, wherein the nutrient-rich medium comprises 50% glycerol, the suitable conditions further comprising glycerol added to the culture at 1.8 mL per hour.

40. The recombinant host cell of claim 33, wherein the non-pathogenic bacterial strain comprises one of BL21-CDAB, HT115-CDAB and Shuffle-CDAB, and wherein the genes of the gene cluster KfiA, KfiB, KfiC, KfiD have been cloned into the recombinant host cell in the order KfiC, kfiD, KfiA, KfiB using one or more vectors.

41. The recombinant host cell of claim 33, wherein the non-pathogenic bacterial strain comprises one of BL21-CDAB, HT115-CDAB and Shuffle-CDAB, and wherein the genes of the gene cluster KfiA, KfiB, KfiC, KfiD have been cloned into the recombinant host cell in the order KfiA, KfiB, KfiC, KfiD using one or more vectors.

42. The recombinant host cell of claim 33, wherein the non-pathogenic bacterial strain comprises one of BL21-CDAB, HT115-CDAB and Shuffle-CDAB, and wherein the genes of the gene cluster KfiA, KfiB, KfiC, KfiD have been cloned into the recombinant host cell individually or with gene pairs KfiAB and KfiCD using one or more vectors, in any order or as a single cluster KfiABCD.

43. The recombinant host cell of claim 33, wherein the suitable conditions comprise one or more temperatures within a range of approximately 14 degrees Celsius to approximately 40 degrees Celsius.

44. The recombinant host cell of claim of claim 43, wherein culturing the recombinant host cell comprises culturing the recombinant host cell at a plurality of the temperatures within the range of approximately 14 degrees Celsius to approximately 40 degrees Celsius.
